SEQUENCE LISTING (19 sequences)

SEQ ID NO: 1 - DNA, 2742 bp, Homo sapiens
  CDS (150)..(1631)
ctagtattctactagaactggaagattgctctccgagttttttttttgttattttgttaaaaaataaaaagcttgagcagcaattcatattactgtcacaggtatttttgctgtgctgtgcaaggtaactctgctagctaagattcacaatgttgaaagcccttttcctaactatgctgactctggcgctggtcaagtcacaggacaccgaagaaaccatcacgtacacgcaatgcactgacggatatgagtgggatcctgtgagacagcaatgcaaagatattgatgaatgtgacattgtcccagacgcttgtaaaggtggaatgaagtgtgtcaaccactatggaggatacctctgccttccgaaaacagcccagattattgtcaataatgaacagcctcagcaggaaacacaaccagcagaaggaacctcaggggcaaccaccggggttgtagctgccagcagcatggcaaccagtggagtgttgcccgggggtggttttgtggccagtgctgctgcagtcgcaggccctgaaatgcagactggccgaaataactttgtcatccggcggaacccagctgaccctcagcgcattccctccaacccttcccaccgtatccagtgtgcagcaggctacgagcaaagtgaacacaacgtgtgccaagacatagacgagtgcactgcagggacgcacaactgtagagcagaccaagtgtgcatcaatttacggggatcctttgcatgtcagtgccctcctggatatcagaagcgaggggagcagtgcgtagacatagatgaatgtaccatccctccatattgccaccaaagatgcgtgaatacaccaggctcattttattgccagtgcagtcctgggtttcaattggcagcaaacaactatacctgcgtagatataaatgaatgtgatgccagcaatcaatgtgctcagcagtgctacaacattcttggttcattcatctgtcagtgcaatcaaggatatgagctaagcagtgacaggctcaactgtgaagacattgatgaatgcagaacctcaagctacctgtgtcaatatcaatgtgtcaatgaacctgggaaattctcatgtatgtgcccccagggataccaagtggtgagaagtagaacatgtcaagatataaatgagtgtgagaccacaaatgaatgccgggaggatgaaatgtgttggaattatcatggcggcttccgttgttatccacgaaatccttgtcaagatccctacattctaacaccagagaaccgatgtgtttgcccagtctcaaatgccatgtgccgagaactgccccagtcaatagtctacaaatacatgagcatccgatctgataggtctgtgccatcagacatcttccagatacaggccacaactatttatgccaacaccatcaatacttttcggattaaatctggaaatgaaaatggagagttctacctacgacaaacaagtcctgtaagtgcaatgcttgtgctcgtgaagtcattatcaggaccaagagaacatatcgtggacctggagatgctgacagtcagcagtatagggaccttccgcacaagctctgtgttaagattgacaataatagtggggccattttcattttagtcttttctaagagtcaaccacaggcatttaagtcagccaaagaatattgttaccttaaagcactattttatttatagatatatctagtgcatctacatctctatactgtacactcacccataacaaacaattacaccatggtataaagtgggcatttaatatgtaaagattcaaagtttgtctttattactatatgtaaattagacattaatccactaaactggtcttcttcaagagagctaagtatacactatctggtgaaacttggattctttcctataaaagtgggaccaagcaatgatgatcttctgtggtgcttaaggaaacttactagagctccactaacagtctcataaggaggcagccatcataaccattgaatagcatgcaagggtaagaatgagtttttaactgctttgtaagaaaatggaaaaggtcaataaagatatatttctttagaaaatggggatctgccatatttgtgttggtttttattttcatatccagcctaaaggtggttgtttattatatagtaataaatcattgctgtacaacatgctggtttctgtagggtatttttaattttgtcagaaattttagattgtgaatattttgtaaaaaacagtaagcaaaattttccagaattcccaaaatgaaccagataccccctagaaaattatactattgagaaatctatggggaggatatgagaaaataaattccttctaaaccacattggaactgacctgaagaagcaaactcggaaaatataataacatccctgaattcaggcattcacaagatgcagaacaaaatggataaaaggtatttcactggagaagttttaatttctaagtaaaatttaaatcctaacacttcactaatttataactaaaatttctcatcttcgtacttgatgctcacagaggaagaaaatgatgatggtttttattcctggcatccagagtgacagtgaacttaagcaaattaccctcctacccaattctatggaatattttatacgtctccttgtttaaaatctgactgctttactttgatgtatcatatttttaaataaaaataaatattcctttagaagatcactctaaaa

SEQ ID NO: 2 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 3 - protein, 493 aa, Rattus norvegicus
MLQTVFLTMLTLALVKSQVTEETITYTQCTDGYEWDPVRQQCKDIDECDIVPDACKGGMKCVNHYGGYLCLPKTAQIIVNNEQPQQETPAAEASSGAATGTIAARSMATSGVIPGGGFIASATAVAGPEVQTGRNNFVIRRNPADPQRIPSNPSHRIQCAAGYEQSEHNVCQDIDECTSGTHNCRLDQVCINLRGSFTCHCLPGYQKRGEQCVDIDECSVPPYCHQGCVNTPGSFYCQCNPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPQNPCQDPYVLTSENRCVCPVSNTMCRDVPQSIVYKYMNIRSDRSVPSDIFQIQATTIYANTINTFRIKSGNENGEFYLRQTSPVSAMLVLVKSLTGPREHIVGLEMLTVSSIGTFRTSSVLRLTIIVGPFSF

SEQ ID NO: 4 - protein, 493 aa, Mus musculus
MLQTLFLTMLTLALVKSQYTEETITYTQCTDGYEWDPIRQQCKDIDECDIVPDACKGGMKCVNHYGGYLCLPKTAQIIVNNEHPQQETPAAEASSGATTGTVAARSMATSGVVPGGGFMASATAVAGPEVQTGRNNFVIRRNPADPQRIPSNPSHRIQCAAGYEQSEHNVCQDIDECTSGTHNCRTDQVCINLRGSFTCQCLPGYQKRGEQCVDIDECTVPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYEVVRSRTCQDINECETTNECREDEMCWNYHGGFRCYPRNPCQDHYVLTSENRCVCPVSNTMCRELPQSIVYKYMSIRSDRSVPSDIFQIQATMIYANTINTFRIKSGNENGEFYLRQTSPVSAMLVLVKSLSGPREYIVDLEMLTVSSIGTFRTSSVLRLTIIVGPFSF

SEQ ID NO: 5 - protein, 448 aa, Mus musculus
MPGLKRILTVTILALWLPHPGNAQQQCTNGFDLDRQSGQCLDIDECRTIPEACRGDMMCVNQNGGYLCIPRTNPVYRGPYSNPYSTSYSGPYPAAAPPVPASNYPTISRPLVCRFGYQMDEGNQCVDVDECATDSHQCNPTQICINTEGGYTCSCTDGYWLLEGQCLDIDECRYGYCQQLCANVPGSYSCTCNPGFTLNDDGRSCQDVNECETENPCVQTCVNTYGSFICRCDPGYELEEDGIHCSDMDECSFSEFLCQHECVNQPGSYFCSCPPGYVLLDDNRSCQDINECEHRNHTCTSLQTCYNLQGGFKCIDPISCEEPYLLIGENRCMCPAEHTSCRDQPFTILYRDMDVVSGRSVPADIFQMQATTRYPGAYYIFQIKSGNEGREFYMRQTGPISATLVMTRPIKGPRDIQLDLEMITVNTVINFRGSSVIRLRIYVSQYPF

SEQ ID NO: 6 - DNA, 21 bp, Artificial
  primer
taacctggatgctgtcgtgga

SEQ ID NO: 7 - DNA, 21 bp, Artificial
  primer
gcccagccagtctgatttgat

SEQ ID NO: 8 - DNA, 21 bp, Artificial
  primer
tgttcctgatgttggtggctt

SEQ ID NO: 9 - DNA, 19 bp, Artificial
  primer
tgtcttggcaaatccggtg

SEQ ID NO: 10 - DNA, 21 bp, Artificial
  primer
aagcctctgtggatatgccca

SEQ ID NO: 11 - DNA, 21 bp, Artificial
  primer
aaccaagaagctgcaggcact

SEQ ID NO: 12 - DNA, 21 bp, Artificial
  primer
gtcccatgatcccttgctaca

SEQ ID NO: 13 - DNA, 20 bp, Artificial
  primer
tgatgcaggcgaagaacttg

SEQ ID NO: 14 - DNA, 21 bp, Artificial
  primer
ccctggctatcagtccaaaca

SEQ ID NO: 15 - DNA, 19 bp, Artificial
  primer
tggcgttgctgatgctctt

SEQ ID NO: 16 - DNA, 21 bp, Artificial
  primer
gaactcattggaggtgcacga

SEQ ID NO: 17 - DNA, 19 bp, Artificial
  primer
tggaacttgtcatccggca

SEQ ID NO: 18 - DNA, 20 bp, Artificial
  primer
gcaatgctggtgcttgtgaa

SEQ ID NO: 19 - DNA, 21 bp, Artificial
  primer
acagagcttgtgcggaaggtt

(19) United States
(12) Patent Application Publication
(10) Pub. No.: US 2006/0094054 A1
(43) Pub. Date: May 4, 2006

(54) Fibulin-3 and uses thereof

(75) Inventors: William P. Schiemann, Denver, CO (US); Allan R. Albig, Denver, CO (US)

Correspondence Address: SHERIDAN ROSS PC, 1560 BROADWAY, SUITE 1200, DENVER, CO, 80202, US

(73) Assignee: National Jewish Medical and Research Center, Denver, CO (US)

(21) Appl. No.: 11/267,942

(22) Filed: Nov. 4, 2005

Related U.S. Application Data
(60) Provisional application No. 60/625,598, filed on Nov. 4, 2004.
(60) Provisional application No. 60/687,129, filed on Jun. 3, 2005.

(51) Int. Cl. C12Q 1/68; G01N 33/574; A61K 48/00

(52) U.S. Cl. 435/6; 435/7.23; 514/44

(57) ABSTRACT

Disclosed is a method and test kit to diagnose tumorigenicity or the presence of tumor cells in a patient by detecting the level of FBLN-3 expression or biological activity in the patient. Also disclosed are methods to identify regulators of tumor cell growth, motility and/or invasion, by identifying regulators of FBLN-3 expression or activity. Methods to identify anti-angiogenic and pro-angiogenic agents are also described, wherein such factors regulate the expression and/or activity of FBLN-3. Finally, the present invention relates to therapeutic methods and reagents for the inhibition of tumor growth and development and/or for the inhibition or promotion of angiogenesis, using FBLN-3, homologues and analogs thereof, and agents that modulate the expression and/or activity of FBLN-3.

### CROSS-REFERENCE TO RELATED APPLICATIONS

This application claims the benefit of priority under 35 U.S.C.§ 119(e) from U.S. Provisional Application Ser. No. 60/625,598, filed Nov. 4, 2004 and from U.S. Provisional Application Ser. No. 60/687,129, filed Jun. 3, 2005. The entire disclosure of each of U.S. Provisional Application Ser. No. 60/625,598 and U.S. Provisional Application Ser. No. 60/687,129 is incorporated herein by reference.

### GOVERNMENT SUPPORT

This invention was made with government support under Grant No. CA095519 and Grant No. CA99321, each awarded by the National Institutes of Health. The government has certain rights to this invention.

### REFERENCE TO SEQUENCE LISTING

This application contains a Sequence Listing submitted on a compact disc, in duplicate. Each of the two compact discs, which are identical to each other pursuant to 37 CFR § 1.52(e)(4), contains the following file: “Sequence Listing”, having a size in bytes of 28 KB, recorded on Nov. 4, 2005. The information contained on the compact disc is hereby incorporated by reference in its entirety pursuant to 37 CFR § 1.77(b)(4).

### FIELD OF THE INVENTION

The present invention generally relates to the use of Fibulin-3, including both long and short forms of Fibulin-3. Specifically, the invention includes the use of Fibulin-3 as a marker for cancer diagnostics and other cancer screening assays, including to monitor the treatment of a patient with cancer, and to the use of short form Fibulin-3 as a cancer therapeutic and/or anti-angiogenesis agent. The present invention also includes the use of long form Fibulin-3 as pro-angiogenic agent (e.g., for stroke, ischemia, etc.) or as a target for anti-angiogenesis therapy. The present invention also relates to methods for identifying regulators of tumorigenicity and angiogenesis.

### BACKGROUND OF THE INVENTION

A key component of physiological tissue development and repair is angiogenesis (Carmeliet, 2000) that, when left unchecked, promotes the pathogenicity of diabetic retinopathy, arthritis, macular degeneration, endometriosis, inflammation, and cancer (Carmeliet and Jain, 2000; Kerbel and Folkman, 2002; Stupack and Cheresh, 2003). Tumor angiogenesis comprises a complex cascade of gene expression and repression that culminates in tumor neovascularization, which facilitates cancer cell proliferation, survival, and ultimately enhances cancer morbidity by establishing an escape route for metastatic cancer cells (Carmetliet and Jain, 2000; Kerbel and Folkman, 2002; Carmeliet, 2003). Thus, identifying novel anti-angiogenic molecules and deciphering their mechanisms of action can be exploited to limit and/or prevent tumor neovascularization, and consequently, to thwart the proliferation, survival, and metastasis of malignant cells.

Six gene products comprise the fibulin family of extracellular matrix (ECM) proteins that function in mediating cell-cell and cell-matrix communication during organogenesis, vasculogenesis, fibrogenesis, and tumorigenesis (Timpl et al., 2003; Argraves et al., 2003). Structurally, fibulins display an elongated configuration of repeating calcium-binding EGF-like modules, followed by a globular C-terminal fibulin-type module. Fibulins are widely expressed and localize to basement membranes, stroma, and ECM fibers, thus providing organization and stabilization to ECM structures by self-associating (e.g., fibulins 1 and 2) (Balbona et al., 1992; Pan et al., 1993), or by interacting with a variety of basement membrane (e.g., laminin, nidogen, and perlecan) and loose connective tissue proteins (e.g., fibronectin, elastin, aggrecan, versican, and endostatin) (Timpl et al., 2003; Argraves et al., 2003).

Fibulin-5 (FBLN-5) was identified originally as a gene product expressed predominantly in developing arteries (Kowal, 1999; Nakamura et al., 1999), and as a novel gene target for TGF-β in fibroblasts and endothelial cells (Schiemann et al., 2002). FBLN-5 now is recognized as a multifunctional ECM protein that (i) binds integrins, apolipoprotein(a), elastin, and EMILIN-1 (Nakamura et al., 1999; Nakamura et al., 2002; Kapetanopoulos et al., 2002; Yanagisawa et al., 2002; Zanetti et al., 2004), and (ii) regulates cell adhesion, proliferation, and motility in a context-specific manner (Nakamura et al., 1999; Schiemann et al., 2002; Nakamura et al., 2002). FBLN-5-deficiency disorganizes elastic fiber architecture leading to lung and vascular malformations, as well as to elastinopathy of the skin reminiscent of cutis laxa syndrome in humans (Nakamura et al., 2002; Yanagisawa et al., 2002), which recently was linked to genetic defects in FBLN-5 (Loeys et al., 2002; Markova et al., 2003). Altered FBLN-5 expression has also been linked to tissue injury and disease, including significant elevations in injured lung (Kuang et al., 2003; Jean et al., 2002) and arteries (Kowal et al., 1999; Nakamura et al., 1999), and dramatic reductions in metastatic malignancies (Schiemann et al., 2002). Thus, FBLN-5 likely contributes to both the initiation and resolution of tissue development, remodeling, and repair.

In U.S. patent application Publication No. 2004-0126788A1, published Jul. 1, 2004 and incorporated herein by reference in its entirety, one of the present inventors disclosed that FBLN-5 expression (i) regulates proliferation in a context-specific manner; (ii) enhances the growth, motility, and invasion of human fibrosarcoma cells; (iii) is aberrant in the majority of metastatic human malignancies, and (iv) stimulates MAP kinases that enhance AP-1 activity stimulated by TGF-β. The inventor is believed to have been the first to demonstrate that FBLN-5 functions as a multifunctional signaling molecule capable of propagating messages between cells or between matrix and cells.

Fibulin-3 (also referred to herein as FBLN-3), which is highly homologous to FBLN-5, has been linked to regulation of macular degeneration (Blackburn, 2003; Guymer, 2002; Marmorstein, 2002; Matsumoto, 2001) and cell proliferation (Lecka-Czernik et al. (1995) Mol. Cell. Biol. 15, 120-128). More specifically, prior to the present invention, researchers had shown that FBLN-3 expression is elevated in fibroblasts undergoing growth arrest or senescence (Lecka-Czernik et al., ibid.), indicating its involvement in inhibiting cell cycle progression. However, microinjection of FBLN-3 mRNA into fibroblasts stimulated DNA synthesis in the injected cells and in their non-injected neighbors. Thus, FBLN-3 regulates cell growth in a context-specific manner via autocrine and paracrine signaling mechanisms. Despite this information, many questions regarding the role of FBLN-3 in mammalian biology remain to be answered, particularly (i) what are the signaling systems/molecules that regulate FBLN-3 expression; (ii) what are the effects of FBLN-3, if any, on cell migration and invasion; and (iii) what are the signaling systems/molecules stimulated by FBLN-3. Moreover, a role for FBLN-3 in angiogenesis or tumorigenesis had not been demonstrated prior to the present invention.

The acquisition of life sustaining gases and nutrients by simple diffusion severely hampers the growth and progression of small, innocuous neoplasms. Indolent tumors overcome this growth impediment by stimulating angiogenesis, a complex cascade of gene expression and repression resulting in the construction of new vasculature networks. Angiogenesis induces new blood vessel formation from preexisting vessels via two distinct phases, activation and resolution. Angiogenesis activation promotes tubulation of quiescent endothelial cells by stimulating their polarization, and their proliferation, invasion, and migration into the tumor microenvironment. Angiogenesis resolution antagonizes the activation phase and instead promotes new vessel maturation and endothelial cell quiescence (Carmeliet, 2000; Carmeliet and Jain, 2000; Kerbel and Folkman, 2002; Hogan and Kolodziej, 2002). Besides facilitating tumor growth and survival, angiogenesis enhances cancer morbidity by providing malignant cells a means for their metastatic spread, which clinically is the most lethal aspect of cancer and the leading cause of cancer-related death (Yoshida et al., 2000). Thus, identifying novel anti-angiogenic molecules and deciphering their mechanisms of action can be exploited to limit and/or prevent tumor neovascularization, and consequently, the proliferation, survival, and metastasis of malignant cells. Therefore, there is a continuing need in the art for novel anti-angiogenic proteins and molecules.

### BRIEF DESCRIPTION OF THE DRAWINGS OF THE INVENTION

FIG. 1A is an alignment of mFBLN-3 (SEQ ID NO:4) to mFBLN-5 (SEQ ID NO:5) (upper case letters with shading indicate amino acid identity and similarity; lower case letters indicate amino acid mismatches).

FIG. 1B is a digitized phosphor image depicting FBLN-3 and ubiquitin expression in paired normal (upper spot) and malignant (bottom spot) tissues.

FIG. 2A is a graph showing FBLN-3 expression during tubulogenesis.

FIG. 2B is a graph showing the resulting GFP expression profiles in MB114 cells infected with control (left panel) or FBLN-3-(right panel) retroviruses.

FIG. 2C is a graph showing the invasion (white bar; n=2), proliferation (black bar; n=3), and angiogenic sprouting (gray bar; n=5) of control- and FBLN-3-expressing MB114 cells.

FIG. 2D shows MB114 cell tubulation proceeding on collagen gels for 5 days in the absence or presence of increasing concentrations of recombinant FBLN-3 (0-50 μg/ml).

FIG. 2E shows the effect of FBLN-3 expression on VEGF165 stimulation of ERK1/ERK2 and p38 MAPK in MB114 cells.

FIG. 3A shows the effect of FBLN-3 and FBLN-5 expression on the expression of MMP-2, MMP-3, TIMP-1, TIMP-2, TIMP-3, and TSP-1.

FIG. 3B shows the effect of FBLN-3 and FBLN-5 expression on MMP-2, MMP-3, and TIMP-3 expression during tubulogenesis.

FIG. 3C shows the effect of FBLN-3 and FBLN-5 expression on MMP-2 activity during tubulogenesis.

FIG. 4A illustrates the angiogenic sprouting of control-, FBLN-3-, or FBLN-5-expressing MB114 cells in complete media (10% serum; black bars), or in serum-free media (SFM; white bars) supplemented with 300 ng/ml bFGF (bFGF; gray bars).

FIG. 4B shows the purity of recombinant FBLNs (2 μg) monitored by Coomassie staining, or by immunoblotting with anti-GST antibodies as indicated.

FIG. 4C illustrates the migration of HMEC-1 cells to fibronectin in the absence or presence of 10 μg/ml of recombinant GST, FBLN-3, or FBLN-5.

FIG. 4D illustrates the adherence of HMEC-1 cells to fibronectin (FN), GST, FBLN-3 (F3), FBLN-5 (F5), or RGE-FBLN-5 (RGE).

FIG. 5A shows the hemoglobin contents corresponding to vascularization observed in harvested Matrigel plugs supplemented with diluent (D), bFGF in presence of (H) recombinant GST, or bFGF in the presence of either 10 μg/ml (L) or 50 μg/ml (H) of recombinant FBLN-3 (F3), FBLN-5 (F5), or RGE-FBLN-5 (RGE).

FIG. 5B shows the vessel densities corresponding to infiltrating vessels in Masson's trichrome-stained Matrigel sections supplemented with diluent (D), bFGF in presence of (H) recombinant GST, or bFGF in the presence of either 10 μg/ml (L) or 50 μg/ml (H) of recombinant FBLN-3 (F3), FBLN-5 (F5), or RGE-FBLN-5 (RGE).

FIG. 6A is a digitized image showing the presence of recombinant FBLN proteins in conditioned-media from GFP (G)-, FBLN-5 (F5)-, or FBLN-3 (F3)-expressing MCA102 fibrosarcoma cells.

FIG. 6B is a graph comparing the invasion of control (GFP)-, FBLN-3-, and FBLN-5-expressing MCA102 fibrosarcoma cells.

FIG. 6C is a graph comparing the tumor mass in mice injected subcutaneously with control (GFP)-, FBLN-3-, or FBLN-5-expressing MCA102 fibrosarcoma cells.

FIG. 6D shows mean (±SE; n=3) blood vessel densities normalized to GFP tumors corresponding to control (GFP)-, FBLN-3-, or FBLN-5-expressing MCA102 fibrosarcoma tumor sections.

FIG. 7 is a graph showing that short and long Fibulin-3 molecules differentially regulate endothelial cell proliferation

FIG. 8 is a graph showing that short and long Fibulin-3 molecules differentially regulate endothelial cell angiogenic sprouting.

FIG. 9 is a graph showing that short and long Fibulin-3 molecules differentially regulate endothelial cell invasion.

FIG. 10 is an alignment of long mFBLN-3 (SEQ ID NO:4) and short mFBLN-3 (positions 107-493 of SEQ ID NO:4).

### SUMMARY OF THE INVENTION

One embodiment of the present invention relates to a method for detecting tumorigenicity in a subject. The method includes the steps of: (a) detecting a level of expression or activity of fibulin-3 (FBLN-3) in a test sample from a subject to be diagnosed; (b) comparing the level of expression or activity of FBLN-3 in the test sample to a baseline level of FBLN-3 expression or activity established from a control sample; and (c) determining whether there is a difference between the levels of FBLN-3 expression. Detection of a statistically significant difference in the level of FBLN-3 expression or activity in the test sample, as compared to the baseline level of FBLN-3 expression or biological activity, indicates a difference in tumorigenicity in the test sample as compared to in the control sample. In one aspect of the invention, detection of a statistically significant difference in the level of FBLN-3 expression or activity in the test sample as compared to the baseline level, with a confidence of p<0.05, indicates that the cells in the test sample have a difference in tumorigenicity as compared to the control sample. In another aspect, detection of an at least about 10% difference in the level of FBLN-3 expression or activity in the test sample as compared to the baseline level, with a confidence of p<0.05, indicates that the cells in the test sample have a difference in tumorigenicity as compared to the control sample. In another aspect, detection of an at least about 1.5 fold difference in the level of FBLN-3 expression or activity in the test sample as compared to the baseline level, with a confidence of p<0.05, indicates that the cells in the test sample have a difference in tumorigenicity as compared to the control sample.

In one aspect of this embodiment, the FBLN-3 is short FBLN-3, including, but not limited to, a short FBLN-3 consisting of amino acids 107493 of SEQ ID NO:2. In another aspect, the FBLN-3 is long FBLN-3, including, but not limited to, FBLN-3 comprising SEQ ID NO:2.

In one aspect of the invention, the step of detecting comprises detecting FBLN-3 mRNA transcription by cells in the test sample, which can include, but is not limited to, a method selected from: polymerase chain reaction (PCR), reverse transcriptase-PCR (RT-PCR), in situ hybridization, Northern blot, sequence analysis, gene microarray analysis, and detection of a reporter gene. In another aspect of the invention, the step of detecting comprises detecting FBLN-3 protein in the test sample, including, but not limited to, a method selected from: immunoblot, enzyme-linked immunosorbant assay (ELISA), radioimmunoassay (RIA), immunoprecipitation, immunohistochemistry and immunofluorescence. In yet another aspect, the step of detecting comprises detecting FBLN-3 biological activity in the test sample by a method including, but not limited to, measuring proliferation of cells expressing FBLN-3, detecting DNA synthesis in cells expressing FBLN-3, detecting MAP kinase activity in cells expressing FBLN-3, detecting MAP kinase activity in the presence of the test sample, and measuring migration and invasion ability of fibroblasts expressing FBLN-3; detecting the ability of FBLN-3 to regulate vascular endothelial growth factor (VEGF) signaling; detecting the ability of FBLN-3 to regulate matrix metalloproteinase (MMP) expression and activity, and detecting the ability of FBLN-3 to regulate tissue inhibitor of metalloproteinase (TIMP) expression.

In one aspect of the invention, the test sample is from a source selected from: breast, kidney, ovary, colon, and uterus. In another aspect, the test sample is a fibroblast cell sample.

In one embodiment of the invention, the test sample is from a patient being diagnosed for cancer, and the baseline level is established from a negative control sample that is non-tumorigenic. Detection of a statistically significant difference in the level of FBLN-3 expression or activity in the test sample, as compared to the baseline level of FBLN-3 expression or biological activity in the negative control sample, indicates the presence of tumor cells in the test sample. In one aspect of this embodiment, when the FBLN-3 expression or biological activity detected in step (a) is statistically significantly different from the baseline level of FBLN-3 expression or activity, the method further comprises: (d) comparing the FBLN-3 expression or activity of the test sample as detected in step (a) to levels of FBLN-3 expression or activity from a panel of tumor-positive control samples, wherein each of the tumor-positive control samples is correlated with a different stage of tumor development; and, (e) identifying a level of FBLN-3 expression or activity from one of the tumor-positive control samples which is statistically significantly most similar to the level of FBLN-3 expression or biological activity detected in step (a), to diagnose a stage of tumor development in the patient. In another aspect of this embodiment, the FBLN-3 is short FBLN-3, and the test sample is not a fibroblast cell sample, and a decrease in the level of short FBLN-3 expression or activity of the test sample as compared to the baseline level of expression or activity indicates the presence of tumor cells in the test sample.

In another embodiment of the invention, the test sample is from a patient who is known to have cancer, and the baseline level comprises a first level of FBLN-3 expression or activity from a previous tumor cell sample from the patient and a second level of FBLN-3 expression or activity established from a negative control cell sample that is non-tumorigenic. In this embodiment, a statistically significant change in the level of FBLN-3 expression or activity in the test sample toward the baseline level established from the negative control cell sample, as compared to the baseline level of expression or activity from the previous tumor cell sample, indicates a reduction in tumor cells in the test sample as compared to the previous tumor cell sample. In one aspect of this embodiment, the method further comprises a step (d) of modifying cancer treatment for the patient if no statistically significant change in the level of FBLN-3 expression or activity in the test sample toward the baseline level established from the negative control cell sample is detected.

In another aspect of the invention, the baseline level is established by a method selected from: (i) establishing a baseline level of FBLN-3 expression or activity in an autologous control sample from the patient, wherein the autologous sample is from a same cell type, tissue type or bodily fluid type as the test sample of step (a); (ii) establishing a baseline level of FBLN-3 expression or activity from at least one previous detection of FBLN-3 expression or activity in a previous test sample from the patient, wherein the previous test sample was of a same cell type, tissue type or bodily fluid type as the test sample of step (a); and, (iii) establishing a baseline level of FBLN-3 expression or activity from an average of control samples of a same cell type, tissue type or bodily fluid type as the test sample of step (a), the control samples having been obtained from a population of matched individuals.

Yet another embodiment of the present invention relates to a test kit for assessing the tumorigenicity of cells in a patient, comprising: (a) a means for detecting FBLN-3 expression or activity in a test sample; and (b) a means for detecting a control marker characteristic of a cell or tissue type that is in the test sample or that is secreted into the test sample by the cell or tissue. In one aspect, the means of (a) is selected from: a hybridization probe of at least about 8 nucleotides that hybridizes under stringent hybridization conditions to a nucleic acid molecule encoding FBLN-3 or a fragment thereof; an oligonucleotide primer for amplification of mRNA encoding FBLN-3 or a fragment thereof; and an antibody that selectively binds to FBLN-3. In one aspect, the means of (b) is selected from: a hybridization probe of at least about 8 nucleotides that hybridizes under stringent hybridization conditions to a nucleic acid molecule encoding the control marker or a fragment thereof; an oligonucleotide primer for amplification of mRNA encoding the control marker or a fragment thereof; and an antibody that selectively binds to the control marker. In another aspect, the means of (a) and (b) are suitable for use in a method of detection selected from the group consisting of immunohistochemistry and immunofluorescence.

Another embodiment of the present invention relates to a method to identify a compound useful for inhibition of tumor growth or malignancy. The method includes the steps of: (a) detecting an initial level of FBLN-3 expression or activity in a tumor cell or soluble product derived therefrom; (b) contacting the tumor cell or soluble product with a test compound; (c) detecting a level of FBLN-3 expression or activity in the tumor cell or soluble product derived therefrom after contact of the tumor cell with the compound; and, (d) selecting a compound that changes the level of FBLN-3 expression or activity in the tumor cell or soluble product therefrom, as compared to the initial level of FBLN-3 expression or activity, toward a baseline level of FBLN-3 expression or activity established from a non-tumor cell, wherein the selected compound is predicted to be useful for inhibition of tumor growth or malignancy.

Yet another embodiment of the present invention relates to a method to identify a compound that regulates angiogenesis, comprising: (a) detecting an initial level of FBLN-3 expression or activity in a cell or soluble product derived therefrom; (b) contacting the cell or soluble product with a test compound; (c) detecting a level of FBLN-3 expression or activity in the cell or soluble product derived therefrom after contact of the cell with the compound; and, (d) selecting a compound that changes the level of FBLN-3 expression or activity in the cell or soluble product therefrom, as compared to in the absence of the compound or as compared to the initial level of FBLN-3 expression or activity, as a compound that regulates angiogenesis. In one aspect of this embodiment, the FBLN-3 is long FBLN-3, and step (d) comprises selecting an agent that increases the expression or activity of long FBLN-3 as an agent that promotes angiogenesis or selecting an agent that decreases the expression or activity of long FBLN-3 as an agent that inhibits angiogenesis. In another aspect of this embodiment, the FBLN-3 is short FBLN-3, and step (d) comprises selecting an agent that increases the expression or activity of short FBLN-3 as an agent that inhibits angiogenesis or selecting an agent that decreases the expression or activity of short FBLN-3 as an agent that promotes angiogenesis.

Another embodiment of the present invention relates to a method to regulate angiogenesis in a tissue of a subject, comprising regulating the expression or biological activity of FBLN-3 in the cells of the tissue. In one aspect, the method inhibits angiogenesis in the tissue of the subject, and the method comprises increasing the expression or biological activity of short FBLN-3 in the cells of the tissue, or decreasing the expression or biological activity of long FBLN-3 in the cells of the tissue. For example, the step of increasing the expression or activity of short FBLN-3 can include administering short FBLN-3 or a biologically active homologue or analog thereof to the patient. Increasing the expression of short FBLN-3 can also include expressing a recombinant nucleic acid molecule encoding short FBLN-3 or a homologue thereof in the tissue of the patient. In another aspect, the method promotes angiogenesis in a tissue of a patient, and the method comprises decreasing the expression or biological activity of short FBLN-3 in the cells of the tissue, or increasing the expression or biological activity of long FBLN-3 in the cells of the tissue. For example, the step of increasing the expression or activity of long FBLN-3 can include administering long FBLN-3 or a biologically active homologue or analog thereof to the patient. The step of increasing the expression of long FBLN-3 can also include expressing a recombinant nucleic acid molecule encoding long FBLN-3 or a homologue thereof in the tissue of the patient.

Yet another embodiment of the present invention relates to a method to reduce tumorigenicity in a patient, comprising regulating the expression or biological activity of FBLN-3 in tumor cells of the patient. In one aspect, the tumor cells are from a tissue selected from: breast, ovary, kidney, colon, and uterus. In one aspect, the method includes administering short FBLN-3 or a biologically active homologue or analog thereof to the patient. In another aspect, the method includes inhibiting the expression or biological activity of long FBLN-3 in the tumor cells. In another aspect, the method includes expressing a recombinant nucleic acid molecule encoding short FBLN-3 or a homologue thereof in the tissue of the patient.

Another embodiment of the present invention relates to a method to reduce tumorigenicity of a fibrosarcoma in a patient, comprising regulating the expression or biological activity of FBLN-3 in fibrosarcoma cells of the patient. In one aspect, the method includes decreasing the expression or biological activity of short FBLN-3 in fibrosarcoma cells of the patient.

### DETAILED DESCRIPTION OF THE INVENTION

The present invention generally relates to the discovery by the present inventors of novel uses of FBLN-3, and in particular, of novel and opposite uses of two splice variants of FBLN-3, denoted herein as the “long form of Fibulin-3” or “long FBLN-3” and the “short form of Fibulin-3” or “short FBLN-3” (described in more detail below). In particular, the present invention describes the use of FBLN-3 (short or long) as a marker for cancer diagnostics and other cancer screening assays, including to monitor the treatment of a patient with cancer, and to the use of short FBLN-3 as a cancer therapeutic and/or anti-angiogenesis agent. The present invention also describes the use of long FBLN-3 as a pro-angiogenic agent, and the inhibition of long FBLN-3 as a cancer therapeutic strategy and/or as an anti-angiogenesis strategy.

First, the present inventors have discovered that FBLN-3, and specifically, short FBLN-3, has aberrant expression in various tumor cells (primarily downregulated). Therefore, a change in short FBLN-3 expression or activity in a cell as compared to a normal control for the cell is a useful marker for cancer diagnostic assays and cancer screening assays. In addition, the present invention is directed to the up- or down-regulation, restoration, or replacement of short FBLN-3 in cells of a patient by protein administration, drug administration or gene therapy as a method of treating cancer and as a method of inhibiting angiogenesis.

The inventors also show for the first time that short FBLN-3 mRNA expression, like that of FBLN-5, is downregulated in human tumors, and that short FBLN-3 antagonizes angiogenic sprouting by (i) inhibiting vascular endothelial growth factor (VEGF) signaling, (ii) decreasing matrix metalloproteinase (MMP) expression and activity, and (iii) increasing tissue inhibitor of metalloproteinase (TIMP) expression. The present inventors' findings demonstrate that short FBLN-3, like FBLN-5, functions as a multifunctional signaling molecule capable of propagating messages between cells or between matrix and cells.

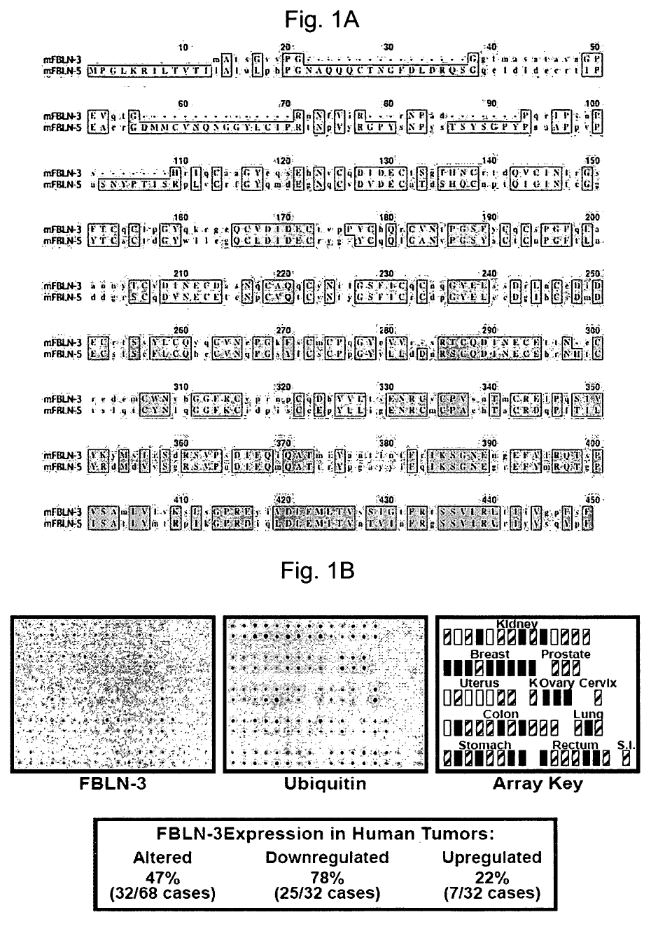

FBLN-3 is homologous to FBLN-5 (see FIG. 1A) and, like FBLN-5, regulates cell proliferation in a context-specific manner (Lecka-Czernik et al., 1995). FBLN-3 was isolated originally as an upregulated gene product expressed in senescent cells (Lecka-Czernik et al., 1995), and again independently as a gene associated with Malattia Leventinese, an inherited form of macular degeneration characterized by the formation of drusen deposits that arise from the aberrant accumulation of misfolded mutant FBLN-3 proteins [i.e., Arg345Trp substitution; (Marmorstein et al., 2002; Matsumoto and Traboulsi, 2001; Stone et al., 1999)]. Likewise, missense FBLN-5 mutations have recently been identified and associated with the development of age-related macular degeneration (Stone et al., 2004). Interestingly, inappropriate ocular angiogenesis plays a prominent role in causing irreversible blindness, including that mediated by macular degeneration (Adamis et al., 2999). The present inventors' findings showing that FBLNs 3 and 5 both inhibit endothelial cell activities in vitro and angiogenesis in vivo, indicate that mutation-induced structural anomalies may inactivate the anti-angiogenic function of FBLNs 3 and 5, thereby contributing to macular degeneration development. Patients housing FBLN-3 or FBLN-5 mutations may be more susceptible to other angiogenic pathologies, particularly cancer.

However, a direct function for FBLN-3 in regulating tumorigenesis and angiogenesis has not been established prior to the present invention. The present inventors show herein that FBLN-3 expression completely recapitulated the anti-angiogenic activities of FBLN-5 in endothelial cells in vitro, and that tumorigenesis downregulates FBLN-3 expression in human malignancies. The inventors further show that FBLNs 3 and 5 both target endothelial cell expression of matrix metalloproteinases (MMPs), tissue inhibitor of metalloproteinases (TIMPs), and thrombospondin-1 (TSP-1), thereby potentially reducing ECM proteolysis and remodeling. Finally, the present inventors demonstrate for the first time that FBLN-3, like FBLN-5, antagonizes vessel development and angiogenesis stimulated by basic fibroblast growth factor (bFGF) both in vitro and in vivo, as well as the neovascularization and growth of fibrosarcomas implanted subcutaneously in mice. Collectively, the studies described herein establish novel mechanisms whereby FBLN-3 antagonizes endothelial cell activities and angiogenesis both in vitro and in vivo, resulting in diminished tumor angiogenesis and growth in mice. The present inventors' study further indicates that the antiangiogenic activities of FBLN-3 can be exploited to prevent the growth and metastasis of human malignancies.

The present inventors have found that tumorigenesis dramatically downregulates short FBLN-3 expression in nearly half of all human malignancies surveyed (FIG. 1B). Moreover, the loss of FBLN-3 in tumor microenvironments is also observed with FBLN-5 (U.S. patent application Publication No. 2004-0126788A1; Schiemann et al., 2002), and both FBLNs are capable of inhibiting or stimulating cell proliferation in a cell- and context-specific manner (U.S. patent application Publication No. 2004-0126788A1; Schiemann et al., 2002; Albig and Schiemann, 2004; Lecka-Czernik et al., 1995). The findings presented herein demonstrate the angiostatic function for short FBLN-3 and indicate that short FBLN-3 acts to suppress the formation and progression of human malignancies.

Indeed, based on the overlapping expression and activity profiles attributed to FBLN family members, without being bound by theory, the present inventors believe that additional FBLNs other than FBLNs 3 and 5 will also regulate angiogenesis and vascular development. This notion is supported by the spatiotemporal manner in which FBLNs 1 and 2 are expressed during cardiovascular development, which suggests involvement of these FBLNs in formation of the aortic arch, and in endocardial and epicardial structures (Timpl et al., 2003; Argraves et al, 2003; Chu and Tsuda, 2004). Along these lines, neonatal and adult endothelial and vascular smooth muscle cells produce FBLNs, which incorporate into vascular basement membranes, elastic laminae, and vessel walls where they likely create an angiostatic environment (Timpl et al., 2003; Argraves et al, 2003; Chu and Tsuda, 2004). Indeed, FBLN-5-deficient mice exhibit exaggerated vascular remodeling following carotid artery ligation and injury, indicating that FBLN-5 functions to limit vascular remodeling and repair in injured vessels. Moreover, vascular smooth muscle cells isolated from these animals display elevated proliferative and migratory activities as compared to those of their FBLN-5-expressing counterparts ((Spencer et al., 2005). In addition, FBLNs 1 and 2 interact physically with the angiogenesis inhibitor, endostatin, presumably mediating its localization within vessel microenvironments (Timpl et al., 2003; Sasaki et al., 1998). Finally, FBLN-1-deficiency in mice elicits perinatal lethality as a consequence of neural and epidermal hemorrhaging of irregularly shaped and aberrantly dilated vessels formed by morphologically abnormal endothelial cells (Kostka et al., 2001). Using the information provided by the present invention, one can now identify which FBLNs (and in which context) suppress and/or promote angiogenesis and examine how manipulations of FBLN expression in mice mimics cancer formation and progression in humans.

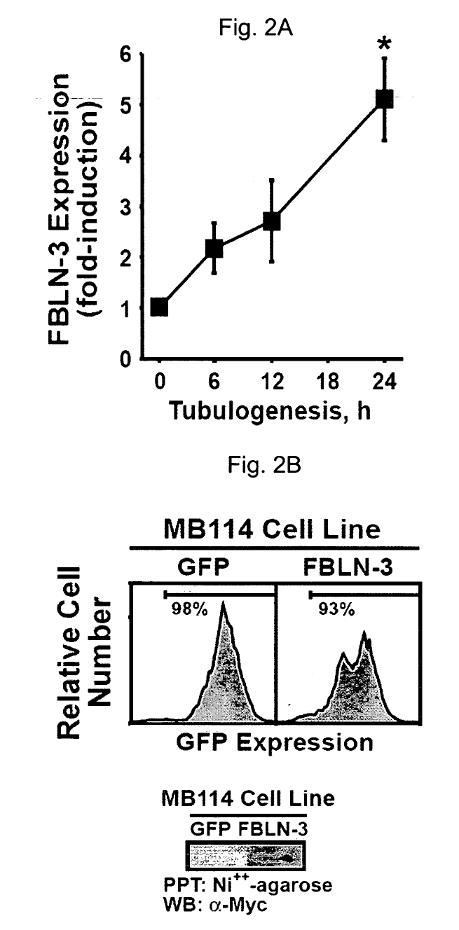

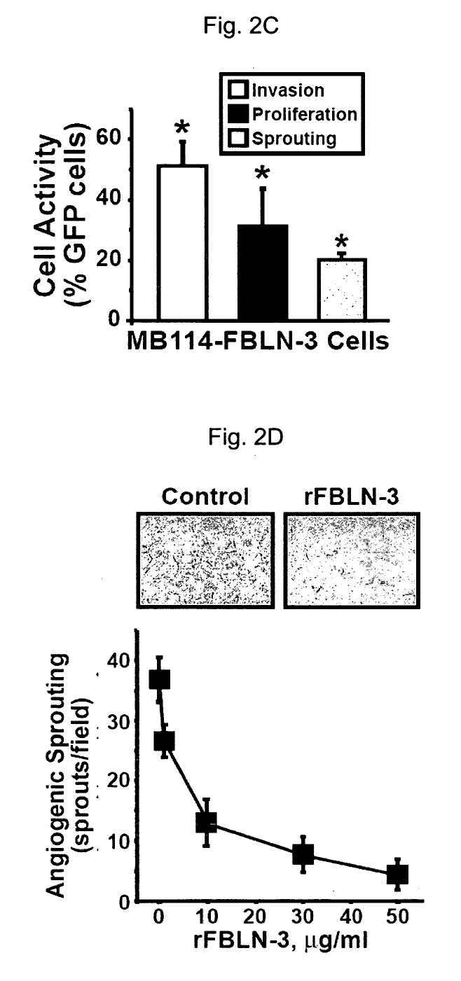

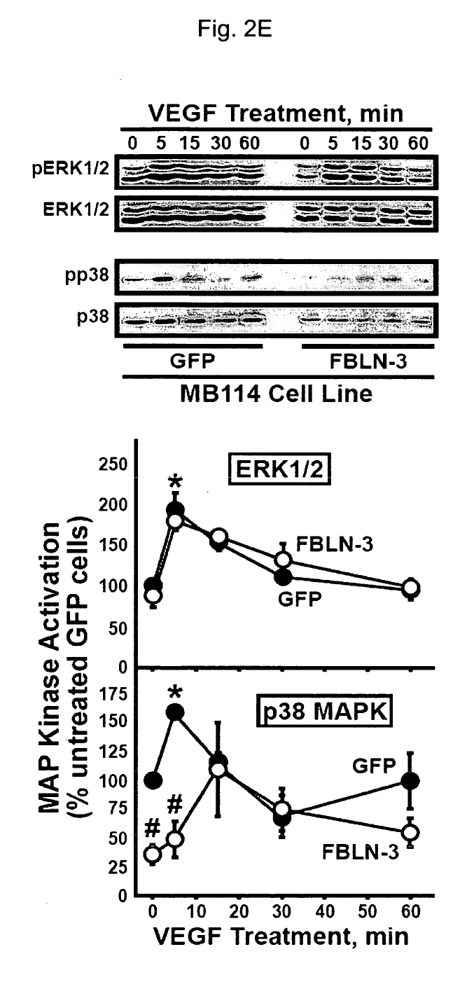

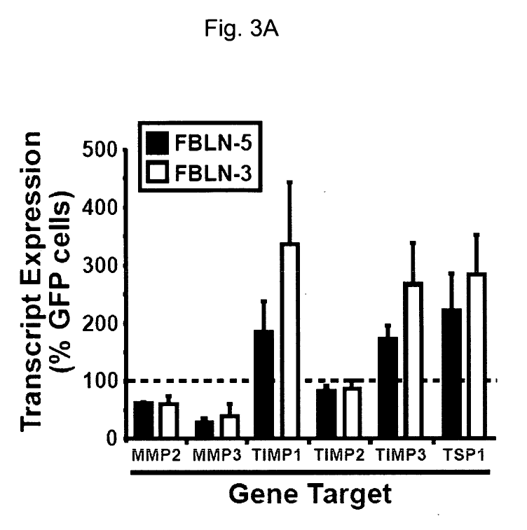

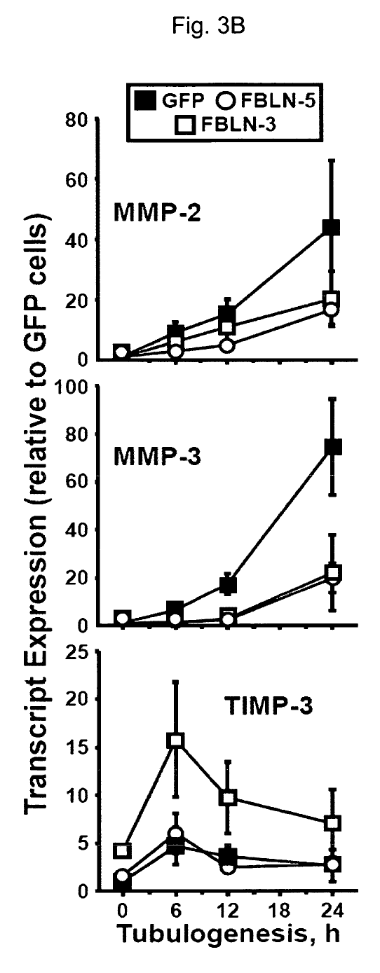

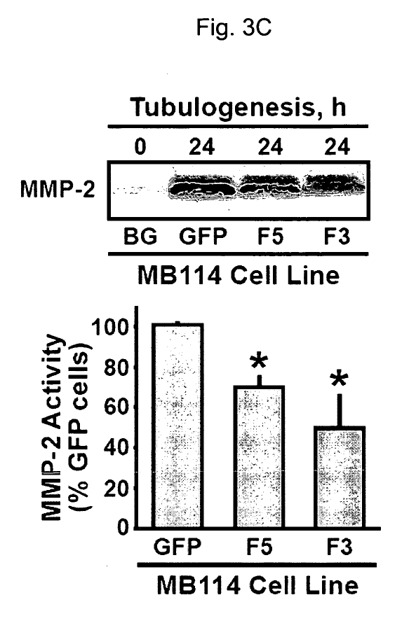

The present inventors' study presents the first evidence that short FBLN-3 is functionally similar to FBLN-5 in its ability to antagonize angiogenesis. For instance, in addition to inhibiting endothelial cell proliferation and invasion, both FBLNs attenuate endothelial cell responses to VEGF, particularly its ability to activate MAP kinases (FIG. 2; U.S. patent application Publication No. 2004-0126788A1; Albig and Schiemann, 2004). Activated p38 MAPK induces actin cytoskeletal rearrangements necessary for endothelial cell migration stimulated by VEGF, whereas activated ERK1/ERK2 induces MMP expression stimulated by VEGF (Carmeliet, 2000; Folkman and Shing, 1992; Terman and Dougher-Vermazen, 1996). Thus, FBLNs 3 and 5, by inhibiting VEGF stimulation of MAP kinases, may reduce endothelial cell invasion and migration requisite for angiogenesis activation. The present inventors' findings indicate that this inhibitory state is likely magnified and/or maintained by the ability of FBLNs 3 and 5 to (i) repress MMP-2 and -3 expression, while simultaneously inducing that of TIMP-1 and -3 in resting and tubulating endothelial cells (FIG. 3); and (ii) induce TSP-1 expression, which antagonizes angiogenesis by stimulating endothelial cell apoptosis (Guo et al., 1997), and by inhibiting MMP-9 activation (Rodriguez-Manzaneque et al., 2001). Collectively, the present inventors' work previously and in the present invention implicates FBLNs 3 (short form) and 5 as novel suppressors of endothelial cell ECM remodeling and proteolysis, and thus creators of endothelial and tumor microenvironments that inhibit vessel formation by promoting angiogenesis resolution.

Despite the shared angiostatic activities between FBLNs 3 (short form) and 5, these FBLNs exhibit distinct expression profiles over the first 24 h of tubulation by MB114 cells. Whereas tubulating MB114 cells dramatically downregulate FBLN-5 expression, that of FBLN-3 is induced significantly (FIG. 2). The reasons underlying the counterintuitive expression of short FBLN-3 are currently unknown, but may be related to distinct spatiotemporal requirements for each FBLN to negate vessel development in MB114 cells. Recently, Bell and colleagues (Bell et al., 2001) found that tubulating human umbilical vein endothelial cells rapidly downregulate their expression of FBLN-3 (by 8 hr), which then returns to basal levels by 24 hr. Although these researchers never examined the function of FBLN-3 in tubulating human umbilical vein endothelial cells, the discordant FBLN-3 expression profiles observed between the present inventors' respective investigations may result from differences in the cell types studied, particularly their kinetics of tubule formation. Alternatively, differences in FBLN-3 expression may reflect the detection of distinct alternatively spliced FBLN-3 mRNAs, of which five unique FBLN-3 transcripts having distinct 5′ sequences have been described (i.e., transcripts 1a, 1b, 2, 3, and 4 (Lecka-Czernik et al., 1995)). Indeed, the present inventors' real-time PCR primers amplify a C-terminal fragment of the short form of FBLN-3, a region common to all FBLN-3 splice variants, while those employed by Bell and colleagues (Bell et al., 2001) selectively amplify FBLN-3 transcript 2. Thus, it is possible that quiescent and tubulating endothelial cells may differentially express FBLN-3 splice variants capable of mediating distinct activities in endothelial cells. In support of this position, the present inventors have determined that stable endothelial cell expression of the long form of FBLN-3 (i.e., transcript 1a, represented herein by SEQ ID NO:4 in mice or SEQ ID NO:2 in humans), which contains an additional N-terminal 106 amino acids not found in its short counterpart (represented herein by positions 107-493 of SEQ ID NO:4 in mice or by positions 107-493 of SEQ ID NO:2 in humans) studied herein, stimulates MB114 cell angiogenic sprouting in vitro (see Example 6).

Further, TIMP-3 mutations have been associated with the development of Sorsby fundus dystrophy (Langton et al., 2000; Tabata et al., 1998; Weber et al., 1994), an early onset inherited form of macular degeneration that resembles age-related macular degeneration. Interestingly, VEGF stimulation of angiogenesis is prevented by the physical association of the VEGF KDR receptor with TIMP-3, which antagonizes VEGF binding (Qi et al., 2003). The present inventors observed short FBLN-3 to stimulate endothelial cell expression of TIMP-3 (FIG. 3B), which interacts physically with FBLN-3 via its C-terminal FBLN-type module (Klenotic et al., 2004). These findings raise the possibility that inhibition of VEGF signaling by TIMP-3 requires its association with FBLN-3, and conversely, that short FBLN-3-mediated inhibition of VEGF signaling in endothelial cells requires FBLN-3:TIMP-3 complex formation.

In summary, the present inventors have established short FBLN-3 as a novel antagonist of angiogenesis, one that is biologically similar with FBLN-5 both in vitro and in vivo. In addition, the present inventors show for the first time that short FBLN-3, like FBLN-5, prevents vessel development and angiogenesis in genetically normal mice, indicating that these ECM proteins possess cancer chemopreventive activities. Accordingly, the present inventors demonstrate for the first time that short FBLN-3, like FBLN-5, inhibits the growth and neovascularization of tumors arising from implanted MCA102 fibrosarcoma cells. Short FBLN-3 tumor therapy is expected to alter the growth, invasion, and angiogenesis of established tumors. In addition, it is believed that the loss of short FBLN-3, as well as FBLN-5, from tumor microenvironments promotes cancer progression by alleviating the constraints to angiogenesis. Therefore, one can now identify the minimal FBLN-3 determinants necessary in mediating its antiangiogenic activities. Indeed, the results of these studies will enable the engineering of novel FBLN-3 proteins or peptide mimetics whose growth and motility promoting activities are fully separated from their angiogenic suppressing activities, thereby improving their anti-angiogenic effectiveness.

The precise molecular mechanism(s) whereby fibulins 5 and 3 (short form) antagonize angiogenesis is currently unknown. Based on the inventors' current understanding of fibulin biology, four potential scenarios whereby FBLN-5 or FBLN-3 (short) antagonize angiogenic sprouting are envisioned. Importantly, these scenarios need not be mutually exclusive. First, the inventors' finding that ablation of the integrin-binding RGD motif in FBLN-5 significantly enhances its anti-angiogenic activities suggests that FBLN-5 may mediate pro- and anti-angiogenic functions by interacting with two distinct receptors in a concentration-dependent manner. For instance, low FBLN-5 concentration is predicted to favor high-affinity binding to pro-angiogenic integrins (i.e., high-affinity, low occupancy), while high FBLN-5 concentration is predicted to favor low-affinity binding to unknown anti-angiogenic receptors (i.e., low-affinity, high occupancy). Second, RGD-mediated binding of FBLN-5 to integrins may tether and/or sequester FBLN-5 from its anti-angiogenic receptor. Third, like fibulins 1 and 2, FBLN-5 or FBLN-3 (short form) may interact physically with angiogenesis inhibitors (e.g., endostatin), thereby enhancing their anti-angiogenic activities in endothelial cells. Finally, by binding elastin, FBLN-5 or FBLN-3 (short form) may protect elastin from proteolytic cleavage, thereby preventing the release of chemotactic peptides that enhance normal and cancer cell motility (Lapis et al., Semin Cancer Biol 2002; 12:209-17).

Finally, as discussed above, the present inventors have discovered that stable endothelial cell expression of long FBLN-3, which contains an additional N-terminal 106 amino acids not found in short FBLN-3, stimulates MB114 cell angiogenic sprouting in vitro. Therefore, the present invention encompasses the use of long FBLN-3 as both a pro-angiogenic agent and also as a target for inhibition in anti-angiogenesis therapy. In addition, the invention contemplates that at least some tumor cells may further show abnormal, enhanced expression of long FBLN-3, making long FBLN-3 a further biomarker for cancer diagnostics described herein. Without being bound by theory, one explanation for the opposing functions of long FBLN-3 and short FBLN-3 may be that expression of the isoforms, or the ratio of the different isoforms, is differentially regulated in different cell types and/or in different microenvironments. For example, cancer and other diseases that are dependent upon inappropriate angiogenesis may cause alterations in the expression various FBLN-3 splice variants. In addition, as a result of the present inventors' discovery, various FBLN-3 isoforms may be used clinically to detect angiogenic disease development in humans, as well as to predict the clinical course of developing disease.

In terms of disease development, without being bound by theory, the present inventors expect that the inappropriate absence or presence of FBLN-3 (long or short form) in cell microenvironments will elicit profound effects on a variety of cellular activities and processes, particularly those involved in tissue development, remodeling, and repair.

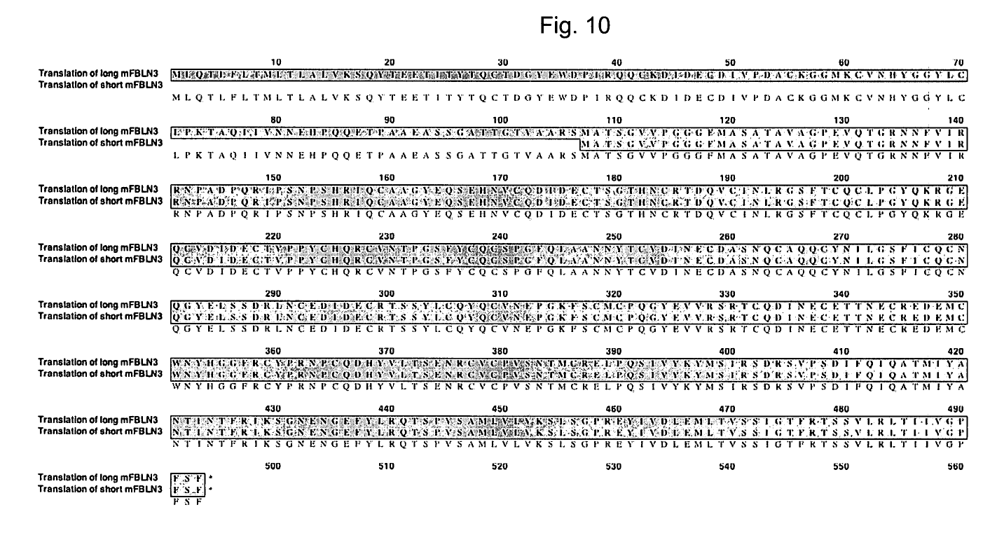

Five different splice variants of FBLN-3 have been described, although only two of the five proteins encoded by the proposed variants have been detected as genuine protein products (Lecka-Czernik et al., supra). These two variants are referenced herein as short FBLN-3 (transcript 4 in Lecka-Czernik et al.) and long FBLN-3 (transcript la in Lecka-Czernik et al.). As discussed above, short FBLN-3 differs from long FBLN-3 by a truncation of 106 amino acids from the N-terminus. According to the present invention, “long FBLN-3” or the “long form of FBLN-3” refers to the entire 493 amino acid FBLN-3 protein (or transcript la from Lecka-Czernik et al.), having a predicted molecular mass of about 56.4 kDa, the amino acid sequence of the human form of which is found in GenBank Accession No. GI:9973182, incorporated herein by reference in its entirety. The amino acid sequence for human long FBLN-3 is represented herein by SEQ ID NO:2. “Short FBLN-3” or the “short form of FBLN-3” refers to the 387 amino acid FBLN-3 protein (or transcript 4 from Lecka-Czernik et al.), having a predicted molecular mass of about 43.1 kDa, the amino acid sequence of the human form of which is represented by amino acids 107-493 of the amino acid sequence in GenBank Accession No. GI:9973182, which is represented herein by positions 107 to 493 of SEQ ID NO:2. GenBank Accession No. NM_{—}004105, also incorporated herein by reference in its entirety, contains the complete mRNA sequence encoding FBLN-3, with the start positions of long FBLN-3 and short FBLN-3 noted. The nucleic acid sequence encoding long FBLN-3 is represented herein by SEQ ID NO:1 (cds located at positions 150-1631 of SEQ ID NO:1). The nucleic acid sequence encoding short FBLN-3 (transcript 4 from Lecka-Czernik et al.) is represented herein by positions 468-1631 of SEQ ID NO:1. The amino acid sequence for the long form of rat FBLN-3, represented herein by SEQ ID NO:3, is found in GenBank Accession No. GI:34879642 or GI:9973135, each of which is also incorporated herein by reference in its entirety. The short form of rat FBLN-3 is represented by positions 107 to 493 of SEQ ID NO:3. The amino acid sequence for the long form of murine FBLN-3, represented herein by SEQ ID NO:4, is found in GenBank Accession No. GI: 62510691, which is incorporated herein by reference in its entirety. The short form of murine FBLN-3 is represented by positions 107 to 493 of SEQ ID NO:4. An alignment of the amino acid sequence for murine short FBLN-3 and murine long FBLN-3 is provided in FIG. 10.

Reference generally to “FBLN-3” herein can be a reference to either form of FBLN-3. Where it is important to designate the particular form, specific reference will be made to the short or long form or will be otherwise inferred from the context of the discussion.

One embodiment of the present invention relates to a method (i.e., an assay) for assessing the presence of a tumor in an individual. In one aspect of this embodiment, the method includes the steps of: (a) detecting a level of expression or activity of short FBLN-3 in a test sample from a subject (individual, patient, animal, person) to be diagnosed; and (b) comparing the level of expression or activity of short FBLN-3 in the test sample to a baseline level of short FBLN-3 expression or activity established from a control sample. It is noted that the present inventors have determined that expression of FBLN-3 is primarily downregulated in various tumor cells, and this FBLN-3 is the form referred to herein as short FBLN-3. If desired, one may confirm that short and not long FBLN-3 is detected, but his is not required. Both the long and short form of FBLN-3 share the C-terminal 387 amino acids. According to the present invention, detection of short FBLN-3 can be achieved by any method that detects the expression of the short form of FBLN-3 (e.g., amino acids 107-493 of any of SEQ ID NO:2, 3 or 4, depending on the subject), but does not require an additional step of confirming that the long or short form is being detected for this assay. Detection of a statistically significant difference in short FBLN-3 expression or activity in the test sample, as compared to the baseline level of short FBLN-3 expression or biological activity, is an indicator of a difference in the tumorigenicity or potential therefore of cells in the test sample as compared to cells in the control sample. As discussed above, expression of short FBLN-3 is cell- and context-specific. Therefore, short FBLN-3 expression or activity could be either upregulated or downregulated in a cell as compared to the control. In most tumor cell types, short FBLN-3 will be downregulated as compared to a normal (non-tumor) cell of the same cell type. Therefore, in one aspect of the invention, detection of reduced short FBLN-3 expression or reduced short FBLN-3 biological activity as compared to the baseline level of short FBLN-3 expression or biological activity, is an indicator of increased tumorigenicity or potential therefore, or the presence of tumor cells, in the test sample. In this aspect of the invention, the cells in the test sample might be from, for example, breast, kidney, colon, ovary, uterus, or a metastatic cancer. However, in another aspect of the invention, particularly when the cells in the test sample are fibroblasts, detection of increased short FBLN-3 expression or increased short FBLN-3 biological activity as compared to the baseline level of short FBLN-3 expression or biological activity, is an indicator of increased tumorigenicity or potential therefore, or the presence of tumor cells, in the test sample. In either case, detection of substantially the same short FBLN-3 expression or biological activity as the baseline control (i.e., differences between sample and baseline control are not statistically significant with a degree of confidence of p<0.05) indicates no significant change or difference in tumorigenicity or the potential therefore by the cell in the test sample (i.e., relative to the baseline control). If the baseline is a normal (non-tumor) test sample, then detection of substantially the same short FBLN-3 expression or activity indicates the absence of tumor cells or tumorigenicity in the test sample. The method of the present invention can be used for any type of tumor wherein short FBLN-3 activity is found to be statistically significantly changed in tumor cells as compared to the corresponding normal cells.

It is recognized that it may ultimately be determined that some cells have aberrant or unusual expression (e.g., increased or upregulated expression) of long FBLN-3 as a result of cancer or other disease and therefore, the detection of long FBLN-3 (e.g., increased expression of long FBLN-3) in this instance as a biomarker of disease, including cancer, is also encompassed by the invention. Therefore, in another aspect of this embodiment of assessing tumorigenicity of cells of the invention, the method includes the steps of: (a) detecting a level of expression or activity of long FBLN-3 in a test sample from a patient to be diagnosed; and (b) comparing the level of expression or activity of long FBLN-3 in the test sample to a baseline level of long FBLN-3 expression or activity established from a control sample. Without being bound by theory, it is believed that expression of long FBLN-3 will be primarily upregulated in various tumor cells. If desired, one may combine the method described above with this method and detect both the short and not long FBLN-3, but this is not required. According to the present invention, detection of long FBLN-3 can be achieved by any method that detects the expression of the long form of FBLN-3 (e.g., amino acids 1-493 of SEQ ID NOs:2, 3 or 4) and therefore particularly includes the detection of at least a fragment of amino acids 1-106 (the N-terminal amino acids, including all or any portion of amino acids 1-106 of the full-length (from the longest transcript) FBLN-3 amino acid sequence) or the nucleotides encoding these amino acids, since this portion of FBLN-3 distinguishes the long FBLN-3 from short FBLN-3. Detection of a statistically significant difference in long FBLN-3 expression or activity in the test sample, as compared to the baseline level of long FBLN-3 expression or biological activity, is an indicator of a difference in the tumorigenicity or potential therefore of cells in the test sample as compared to cells in the control sample. Expression of long FBLN-3 is predicted to be cell- and context-specific, as for short FBLN-3. Therefore, long FBLN-3 expression or activity could be either upregulated or downregulated in a cell as compared to the control. In most tumor cell types, long FBLN-3 is expected to be upregulated as compared to a normal (non-tumor) cell of the same cell type. Therefore, in one aspect of the invention, detection of increased long FBLN-3 expression or increased long FBLN-3 biological activity as compared to the baseline level of long FBLN-3 expression or biological activity, is an indicator of increased tumorigenicity or potential therefore, or the presence of tumor cells, in the test sample. In another aspect of the invention, detection of decreased long FBLN-3 expression or decreased long FBLN-3 biological activity as compared to the baseline level of long FBLN-3 expression or biological activity, is an indicator of decreased tumorigenicity or potential therefore, or the absence of tumor cells, in the test sample. In either case, detection of substantially the same long FBLN-3 expression or biological activity (i.e., differences between sample and baseline control are not statistically significant with a degree of confidence of p<0.05) indicates no significant change or difference in tumorigenicity or the potential therefore in the test sample (i.e., relative to the baseline control). The method of the present invention can be used for any type of tumor wherein long FBLN-3 expression or activity is found to be statistically significantly changed in tumor cells as compared to the corresponding normal cells.

According to the present invention, the phrase “tumorigenicity” refers primarily to the tumor status of a cell or cells (i.e., the extent of neoplastic transformation of a cell, the malignancy of a cell, or the propensity for a cell to form a tumor and/or have characteristics of a tumor), which is a change of a cell or population of cells from a normal to malignant state. Tumorigenicity indicates that tumor cells are present in a sample, and/or that the transformation of cells from normal to tumor cells is in progress, as may be confirmed by any standard of measurement of tumor development. The change typically involves cellular proliferation at a rate which is more rapid than the growth observed for normal cells under the same conditions, and which is typically characterized by one or more of the following traits: continued growth even after the instigating factor (e.g., carcinogen, virus) is no longer present; a lack of structural organization and/or coordination with normal tissue, and typically, a formation of a mass of tissue, or tumor. A tumor, therefore, is most generally described as a proliferation of cells (e.g., a neoplasia, a growth, a polyp) resulting from neoplastic growth and is most typically a malignant tumor. In the case of a neoplastic transformation, a neoplasia is malignant or is predisposed to become malignant. Malignant tumors are typically characterized as being anaplastic (primitive cellular growth characterized by a lack of differentiation), invasive (moves into and destroys surrounding tissues) and/or metastatic (spreads to other parts of the body). As used herein, reference to a “potential for neoplastic transformation”, “potential for tumorigenicity” or a “potential for tumor cell growth” refers to an expectation or likelihood that, at some point in the future, a cell or population of cells will display characteristics of neoplastic transformation, including rapid cellular proliferation characterized by anaplastic, invasive and/or metastatic growth. In the present invention, the expectation or likelihood of tumorigenicity or neoplastic transformation and particularly malignant tumor cell growth (i.e., a positive diagnosis of tumorigenicity) is determined based on a detection of aberrant expression or activity of FBLN-3 (short or long) in a cell as compared to a baseline (i.e., control) level of FBLN-3 expression or biological activity that is considered to be representative of FBLN-3 expression or biological activity in a normal (not neoplastically transformed) cell, as discussed in detail below.

This method of the present invention has several different uses. First, the method can be used to diagnose tumorigenicity, or the potential for tumorigenicity, or simply the presence or absence of tumor cells, in a subject. The subject can be an individual who is suspected of having a tumor, or an individual who is presumed to be healthy, but who is undergoing a routine or diagnostic screening for the presence of a tumor (cancer). The subject can also be an individual who has previously been diagnosed with cancer and treated, and who is now under surveillance for recurring tumor growth. The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified. Therefore, in the present invention, a positive diagnosis (i.e., a positive assessment) of tumor growth or tumorigenicity (i.e., malignant or inappropriate cell growth or neoplastic transformation), or the potential therefor, means that the indicators (e.g., signs, symptoms) of tumor presence and/or growth according to the present invention (i.e., a change in FBLN-3 expression or biological activity as compared to a baseline control) have been identified in the sample obtained from the subject. Such a subject can then be prescribed treatment to reduce or eliminate the tumor growth. Similarly, a negative diagnosis (i.e., a negative assessment) for tumor growth or a potential therefore or the absence of tumor cells means that the indicators of tumor growth or tumor presence or a likelihood of developing tumors as described herein (i.e., a change in FBLN-3 expression or biological activity as compared to a baseline control) have not been identified in the sample obtained from the subject. In this instance, the subject is typically not prescribed any treatment, but may be reevaluated at one or more timepoints in the future to again assess tumor growth. Baseline levels for this particular embodiment of the method of assessment of tumorigenicity of the present invention are typically based on a “normal” or “healthy” sample from the same bodily source as the test sample (i.e., the same tissue, cells or bodily fluid), as discussed in detail below.

In a second embodiment, the method of the present invention can be used more specifically to “stage” a tumor in a subject. Therefore, the subject can be diagnosed as having a tumor, tumor growth, or potential therefore by the method as discussed above, or by any other suitable method-(e.g., physical exam, X-ray, CT scan, blood test for a tumor antigen, surgery), and then (or at the same time, when the present method is also used as a diagnostic), the method of the present invention can be used to determine the stage of progression of tumor growth or tumor presence in an individual. For most cancer types, standard staging criteria exist and are known in the art. For example, in breast tumors, there are five different general stages of tumor development which are known and acknowledged in the art as stages 0, I, II, III and IV (although these stages can be grouped into more complex subgroups based on more specific indicators). In this embodiment of the method of the present invention, the FBLN-3 expression and/or biological activity in the subject sample is compared to a panel of several different “baseline” levels of FBLN-3 expression or biological activity, wherein each baseline level represents a previously established level for a given stage of the cancer being diagnosed. For example, for a breast tumor staging assay, baseline levels of FBLN-3 expression and/or biological activity can be established for Stages I, II, III and IV of breast tumor cells (e.g., using an average level determined from a random sampling of tumors from different patients at the various stages). Therefore, in this embodiment, the level of expression of FBLN-3 expression or biological activity in the subject sample is compared to the various baseline levels corresponding to the different stages of tumor growth to identify the baseline level that is statistically closest to the level of FBLN-3 expression or biological activity detected in the subject. The ability to “stage” a tumor in the method of the present invention allows the physician to more appropriately prescribe treatment for the subject.

In a third embodiment of this method of the present invention, the method is used to monitor the success, or lack thereof, of a treatment for cancer in a patient that has been diagnosed as having cancer. In this embodiment, the baseline level of FBLN-3 expression or biological activity typically includes the previous level of FBLN-3 expression or biological activity in a sample of the patient's tumor, so that a new level of FBLN-3 expression or biological activity can be compared to determine whether tumor cell growth or the presence of tumor cells in a sample is decreasing, increasing, or substantially unchanged as compared to the previous, or first sample (i.e., the initial sample which presented a positive diagnosis). In addition, or alternatively, a baseline established as a “normal” or “healthy” (non-tumor) level of FBLN-3 expression or biological activity can be used in this embodiment, particularly to determine in what manner FBLN-3 expression is regulated in tumors for the given cell type. This embodiment allows the physician to monitor the success, or lack of success, of a treatment that the patient is receiving for cancer, and can help the physician to determine whether the treatment should be modified. In one embodiment of the present invention, the method includes additional steps of modifying cancer treatment for the patient based on whether an increase or decrease in tumor cell growth is indicated by evaluation of FBLN-3 expression and/or biological activity in the patient.

The first step of the method of the present invention includes detecting FBLN-3 (short or long) expression or biological activity in a test sample from a subject. According to the present invention, the term “test sample” can be used generally to refer to a sample of any type which contains cells or products that have been secreted from cells (i.e., FBLN-3 is a secreted protein and so one can evaluate a cell supemate, bodily fluid or other media into which FBLN-3 may have been secreted by a cell) to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. A cell sample can also be processed to obtain a soluble product therefrom, such as a supernatant or lysate from the cell that would contain the soluble FBLN-3 protein. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for FBLN-3 expression or biological activity and/or contains the soluble FBLN-3 secreted by cells, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The sample that is most useful in the present invention will be cells, tissues or bodily fluids isolated from a patient by a biopsy or surgery or routine laboratory fluid collection.

Once a sample is obtained from the subject, the sample is evaluated for detection of FBLN-3 expression or biological activity in the cells of the sample. The phrase “FBLN-3 expression” (including as it applies to either form of FBLN-3) can generally refer to FBLN-3 mRNA transcription or FBLN-3 protein translation. Preferably, the method of detecting FBLN-3 expression or biological activity in the subject is the same or qualitatively equivalent to the method used for detection of FBLN-3 expression or biological activity in the sample used to establish the baseline level.

Methods suitable for detecting FBLN-3 transcription (either form) include any suitable method for detecting and/or measuring mRNA levels from a cell or cell extract. Such methods include, but are not limited to: polymerase chain reaction (PCR), reverse transcriptase PCR (RT-PCR), in situ hybridization, Northern blot, sequence analysis, gene microarray analysis (gene chip analysis) and detection of a reporter gene. Such methods for detection of transcription levels are well known in the art, and many of such methods are described in detail in the attached examples, in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid., and Glick et al., ibid. are incorporated by reference herein in their entireties.

Measurement of FBLN-3 transcription is suitable when the sample is a cell or tissue sample; therefore, when the sample is a bodily fluid sample containing cells or cellular extracts, the cells are typically isolated from the bodily fluid to perform the expression assay, or the fluid is evaluated for the presence of secreted FBLN-3 protein.

FBLN-3 expression can also be identified by detection of FBLN-3 translation (i.e., detection of FBLN-3 protein in a sample). Methods suitable for the detection of FBLN-3 protein include any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry and immunofluorescence. Particularly preferred methods for detection of proteins include any single-cell assay, including immunohistochemistry and immunofluorescence assays. Such methods are well known in the art. Furthermore, antibodies against FBLN-3 are known in the art and are described in the public literature and methods for production of antibodies against FBLN-3 are well known in the art.

The term, “FBLN-3 biological activity” or “FBLN-3 activity” refers to any biological action of the FBLN-3 protein, including, but not limited to, regulation of proliferation of cells expressing FBLN-3, regulation of tumor cell growth, regulation of DNA synthesis in cells expressing FBLN-3, regulation of MAP kinase activity in cells expressing FBLN-3, regulation of migration and invasion ability of cells expressing FBLN-3, upregulation of the expression of MMP-2 or MMP-3, downregulation of the expression of TIMP-1, TIMP-3, or TSP-1, regulation of tissue vascularization, and regulation of vessel density in tissues. The specific activities of short FBLN-3 as an anti-tumor and anti-angiogenesis agent have been described in detail above and are also described in detail in the Examples section. Similarly, the specific activity of long FBLN-3 as a pro-angiogenic agent has been described in detail above and are described in the Examples section. Methods to detect FBLN-3 biological activity are known in the art and described in detail in the attached examples and include, but are not limited to, assays for the detection of any of the above-identified activities. Procedures for the isolation and assay of several Fibulin's, including FBLN-3 have been described in the art (e.g., Markova et al., Am J Hum Genet. April 2003;72(4):998-1004; Loeys et al., Hum Mol Genet. Sep. 1, 2002;11(18):2113-8; Kapetanopoulos et al., Mol Genet Genomics 2002 June;267(4):440-6; Schiemann et al., J Biol Chem. Jul. 26, 2002;277(30):27367-77; Midwood et al., Curr Biol. Apr. 16, 2002;12(8):R279-81; Nakamura et al., Nature. Jan. 10, 2002;415(6868):171-5; Yanagisawa et al., Nature. Jan. 10, 2002;415(6868):168-71; Jean et al., J Physiol Lung Cell Mol Physiol. 2002 January;282(1):L75-82; Kowal et al., Cytogenet Cell Genet. 1999;87(1-2):2-3; Nakamura et al., J Biol Chem. Aug. 6, 1999;274(32):22476-83; and Kowal et al., Circ Res. May 28, 1999;84(10):1166-76; each of which is incorporated by reference in its entirety). Other methods for detection of FBLN-3 biological activity, will be known to those of skill in the art and are encompassed by the present invention, and methods for detection of FBLN-3 activity are described in the attached examples.

The method of the present invention includes a step of comparing the level of FBLN-3 expression or biological activity detected in step (a) to a baseline level of FBLN-3 expression or biological activity established from a control sample. According to the present invention, a “baseline level” is a control level, and in some embodiments (but not all embodiments, depending on the method), a normal (healthy, non-tumor) level, of FBLN-3 expression or activity against which a test level of FBLN-3 expression or biological activity (i.e., in the test sample) can be compared. Therefore, it can be determined, based on the control or baseline level of FBLN-3 expression or biological activity, whether a sample to be evaluated for tumor cell growth has a measurable increase, decrease, or substantially no change in FBLN-3 expression or biological activity, as compared to the baseline level. As discussed above, the baseline level can be indicative of different states of cell tumorigenicity or lack thereof, depending on the primary use of the assay. For example, the baseline level can be indicative of the absence of tumor cells or the absence of tumorigenicity that is expected in a normal (i.e., healthy, negative control, non-tumor) cell sample. Therefore, the term “negative control” used in reference to a baseline level of FBLN-3 expression or biological activity typically refers to a baseline level established in a sample from the subject or from a population of individuals which is believed to be normal (i.e., non-tumorous, not undergoing neoplastic transformation, not exhibiting inappropriate cell growth). It is noted that the “negative control” most typically has a higher level of short FBLN-3 expression or activity than would be detected in an experimental cell having inappropriate, increased cell growth, because the short FBLN-3 expression/biological activity and cell growth are inversely related in most tumor cell types. Conversely, a “negative control” with regard to long FBLN-3 is expected to most typically be a lower level of long FBLN-3 expression or activity than would be detected in a cell having inappropriate, increased cell growth, because the long FBLN-3 expression/biological activity and cell growth are expected to be inversely related in most tumor cell types. However, in some cell types (e.g., fibroblasts), the negative control may have a lower level of short FBLN-3 or a higher level of long FBLN-3 expression or activity than the tumor type.

In another embodiment, a baseline can be indicative of a positive diagnosis of tumor cell growth. Such a baseline level, also referred to herein as a “positive control” baseline, refers to a level of FBLN-3 expression or biological activity established in a cell sample from the subject, another patient, or a population of individuals, wherein the sample was believed, based on data for that cell sample, to be neoplastically transformed (i.e., tumorous, exhibiting inappropriate cell growth, cancerous). It is noted that this “positive control” will most typically actually have a lower level of short FBLN-3 expression or activity than in a normal cell, again due to the inverse relationship between short FBLN-3 and cell growth in the majority of tumor cells, and the inverse is expected to be true of long FBLN-3. As discussed above with regard to the negative control, the inverse can be true for some cell types, such as fibroblasts. In one aspect of this embodiment, the baseline can be indicative of a particular stage of tumor cell growth, which will allow a patient's sample to be “staged” (i.e., the stage of the cancer in the patient can be identified). In yet another embodiment, the baseline level can be established from a previous sample from the patient being tested, so that the tumor growth of a patient can be monitored over time and/or so that the efficacy of a given therapeutic protocol can be evaluated over time. Methods for detecting FBLN-3 expression or biological activity are described in detail above.

The method for establishing a baseline level of FBLN-3 expression or activity is selected based on the sample type, the tissue or organ from which the sample is obtained, the status of the subject to be evaluated, and, as discussed above, the focus or goal of the assay (e.g., diagnosis, staging, monitoring). Preferably, the method is the same method that will be used to evaluate the sample in the subject. In a most preferred embodiment, the baseline level is established using the same cell type as the cell to be evaluated.

In one embodiment, the baseline level of FBLN-3 expression or biological activity is established in an autologous control sample obtained from the subject. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, when the goal of the assay is diagnosis of abnormal cell growth, it is desirable to take the control sample from a population of cells, a tissue or a bodily fluid which is believed to represent a “normal” cell, tissue, or bodily fluid, or at a minimum, a cell or tissue which is least likely to be undergoing or potentially be predisposed to develop tumor cells. For example, if the sample to be evaluated is an area of apparently abnormal cell growth, such as a tumorous mass, the control sample is preferably obtained from a section of apparently normal tissue (i.e., an area other than and preferably a reasonable distance from the tumorous mass) in the tissue or organ where the tumorous mass is growing. In one aspect, if a tumor to be evaluated is in the colon, the test sample would be obtained from the suspected tumor mass and the control sample would be obtained from a different section of the colon, which is separate from the area where the mass is located and which does not show signs of uncontrolled cellular proliferation.

In another embodiment, when the goal is to monitor tumor cell growth in a patient, the autologous baseline sample is typically a previous sample from the patient which was taken from an apparent or confirmed tumorous mass, and/or from apparently normal (i.e., non-tumor) tissue in the patient (or a different type of baseline for normal can be used, as discussed below). Therefore, a second method for establishing a baseline level of FBLN-3 expression or biological activity is to establish a baseline level of FBLN-3 expression or biological activity from at least one measurement of FBLN-3 expression or biological activity in a previous sample from the same patient. Such a sample is also an autologous sample, but is taken from the patient at a different time point than the sample to be tested. Preferably, the previous sample(s) were of a same cell type, tissue type or bodily fluid type as the sample to be presently evaluated. In one embodiment, the previous sample resulted in a negative diagnosis (i.e., no tumor cells, or potential to develop tumor cells, was identified). In this embodiment, a new sample is evaluated periodically (e.g., at annual physicals), and as long as the patient is determined to be negative for tumor development, an average or other suitable statistically appropriate baseline of the previous samples can be used as a “negative control” for subsequent evaluations. For the first evaluation, an alternate control can be used, as described below, or additional testing may be performed to confirm an initial negative diagnosis, if desired, and the value for FBLN-3 expression or biological activity can be used thereafter. This type of baseline control is frequently used in other clinical diagnosis procedures where a “normal” level may differ from patient to patient and/or where obtaining an autologous control sample at the time of diagnosis is either not possible, not practical or not beneficial. For example, for a subject who has periodic mammograms, the previous mammograms serve as baseline controls for the mammary tissue of the individual subject. Similarly, for a subject who is regularly screened for prostate cancer by evaluation of levels of prostate cancer antigen (PCA), previous PCA levels are frequently used as a baseline for evaluating whether the individual subject experiences a change.

In another embodiment, the previous sample from a patient resulted in a positive diagnosis (i.e., the presence of tumor cells was positively identified). In this embodiment, the baseline provided by the previous sample is effectively a positive control for tumor cells, and the subsequent samplings of the patient are compared to this baseline to monitor the progress of the tumor growth and/or to evaluate the efficacy of a treatment that is being prescribed for the cancer. In this embodiment, it may also be beneficial to have a negative baseline level of FBLN-3 expression or biological activity (i.e., a normal cell baseline control), so that a baseline for remission or regression of the tumor can be set. Monitoring of a patient's tumor growth or remission can be used by the clinician to modify cancer treatment for the patient based on whether an increase or decrease in cell growth is indicated.

It will be clear to those of skill in the art that some samples to be evaluated will not readily provide an obvious autologous control sample, or it may be determined that collection of autologous control samples is too invasive and/or causes undue discomfort to the patient. In these instances, an alternate method of establishing a baseline level of FBLN-3 expression or biological activity can be used, examples of which are described below.

Another method for establishing a baseline level of FBLN-3 expression or biological activity is to establish a baseline level of FBLN-3 expression or biological activity from control samples, and preferably control samples that were obtained from a population of matched individuals. It is preferred that the control samples are of the same sample type as the sample type to be evaluated for FBLN-3 expression or biological activity (e.g., the same cell type, and preferably from the same tissue or organ). According to the present invention, the phrase “matched individuals” refers to a matching of the control individuals on the basis of one or more characteristics which are suitable for the type of cell or tumor growth to be evaluated. For example, control individuals can be matched with the subject to be evaluated on the basis of gender, age, race, or any relevant biological or sociological factor that may affect the baseline of the control individuals and the patient (e.g., preexisting conditions, consumption of particular substances, levels of other biological or physiological factors). For example, levels of short FBLN-3 expression in the breast of a normal individual (i.e., having breast tissue that is not neoplastically transformed or predisposed to such transformation) may be higher in individuals of a given classification (e.g., elderly vs. teenagers, smokers vs. non-smokers) (although such variation in groups is not currently known). To establish a control or baseline level of FBLN-3 expression or biological activity, samples from a number of matched individuals are obtained and evaluated for FBLN-3 expression or biological activity. The sample type is preferably of the same sample type and obtained from the same organ, tissue or bodily fluid as the sample type to be evaluated in the test patient. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test subject). The values obtained from the control samples are statistically processed using any suitable method of statistical analysis to establish a suitable baseline level using methods standard in the art for establishing such values.

A baseline such as that described above, can be a negative control baseline, such as a baseline established from a population of apparently normal control individuals. Alternatively, as discussed above, such a baseline can be established from a population of individuals that have been positively diagnosed as having cancer, and particularly, cancer of a specified stage, as set forth by the medical community, so that one or more baseline levels can be established for use in staging a cancer in the patient to be evaluated. Therefore, in one embodiment, the baseline level is one or more tumor control samples that is correlated with a particular stage of tumor development for that type of tumor. For example, tumor samples from an appropriate number of individuals which have been diagnosed as having a particular stage of a given cancer (e.g., Stage I colon cancer) are tested for FBLN-3 expression or biological activity. The values obtained from these control samples are statistically processed to establish a suitable baseline level using methods standard in the art for establishing such values, and the baseline is noted as being indicative of that particular stage of cancer. Preferably, a similar value is determined for each of the established stages of the given cancer, so that a panel of baseline values, each representing a different stage of the cancer, is formed. The level of FBLN-3 expression or biological activity in the patient sample is then compared to each of the baseline levels to determine to which baseline the FBLN-3 level of the patient is statistically closest. It will be appreciated that a given patient sample may fall between baseline levels of two different stages such that the best diagnosis is that the patient tumor is at least at the lower stage, but is perhaps in the process of advancing to the higher stage. The data provided by this method can be used in conjunction with current cancer staging methods to assist the physician in the evaluation of the patient and in prescribing suitable treatment for the cancer.

It will be appreciated by those of skill in the art that a baseline need not be established for each assay as the assay is performed but rather, a baseline can be established by referring to a form of stored information regarding a previously determined baseline level of FBLN-3 expression for a given control sample, such as a baseline level established by any of the above-described methods. Such a form of stored information can include, for example, but is not limited to, a reference chart, listing or electronic file of population or individual data regarding “normal” (negative control) or tumor positive (including staged tumors) FBLN-3 expression; a medical chart for the patient recording data from previous evaluations; or any other source of data regarding baseline FBLN-3 expression that is useful for the patient to be diagnosed.

After the level of FBLN-3 expression or biological activity is detected in the sample to be evaluated for tumor cell growth, such level is compared to the established baseline level of FBLN-3 expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in FBLN-3 expression or biological activity over the baseline level, or whether there is no statistically significant difference between the test and baseline levels. After comparing the levels of FBLN-3 expression or biological activity in the samples, the final step of making a diagnosis, monitoring, or staging of the patient can be performed as discussed above. For example, using the method of the invention, one can determine whether tumor growth has occurred or recurred or remitted in an individual, whether a treatment currently being prescribed is successfully controlling the tumor growth, and one may even detect the stage of development of a given tumor.

As discussed above, a positive diagnosis indicates that increased cell growth, and possibly tumor cell growth (neoplastic transformation), has occurred, is occurring, or is statistically likely to occur in the cells or tissue from which the sample was obtained. In order to establish a positive diagnosis, the level of FBLN-3 activity is modulated (increased or decreased, depending on the cell or tissue type) over the established baseline by an amount that is statistically significant (i.e., with at least a 95% confidence level, or p<0.05). Preferably, detection of at least about a 10% change in FBLN-3 expression or biological activity in the sample as compared to the baseline level results in a positive diagnosis of increased cell growth for said sample, as compared to the baseline. More preferably, detection of at least about a 30% change in FBLN-3 expression or biological activity in the sample as compared to the baseline level results in a positive diagnosis of increased cell growth for said sample, as compared to the baseline. More preferably, detection of at least about a 50% change, and more preferably at least about a 70% change, and more preferably at least about a 90% change, or any percentage change between 5% and higher in 1% increments (i.e., 5%, 6%, 7%, 8% . . . ) in FBLN-3 expression or biological activity in the sample as compared to the baseline level results in a positive diagnosis of increased tumorigenicity for said sample. In one embodiment, a 1.5 fold change in FBLN-3 expression or biological activity in the sample as compared to the baseline level results in a positive diagnosis of increased tumorigenicity for said sample. More preferably, detection of at least about a 3 fold change, and more preferably at least about a 6 fold change, and even more preferably, at least about a 12 fold change, and even more preferably, at least about a 24 fold change, or any fold change from 1.5 up in increments of 0.5 fold (i.e., 1.5, 2.0, 2.5, 3.0 . . . ) in FBLN-3 expression or biological activity as compared to the baseline level, results in a positive diagnosis of increased tumorigenicity for said sample.

Once a positive diagnosis is made using the present method, the diagnosis can be substantiated, if desired, using any suitable alternate method of detection of tumor cell growth, including pathology screening, blood screening for tumor antigens, and surgery. In one embodiment of the present invention, the method can include an additional step of confirming the diagnosis of tumor cell growth using such an alternate form of detection of neoplastic transformation such as surgery, tumor antigen screening, biopsy and/or pathology/histology. A positive diagnosis of tumor cell growth in an individual allows for the commencement of appropriate treatment protocols. Since the method of the present invention is useful for the early detection of inappropriate cell growth in an individual, treatment protocols are expected to be more effective and result in prolonged survival rates.

Yet another embodiment of the present invention relates to a test kit for diagnosing the presence of tumor cells or a potential for tumor cell growth in a patient. The test kit includes: (a) a means for detecting FBLN-3 expression or activity in a test sample; and (b) a means for detecting a control marker characteristic of a cell type in the test sample.

This test kit, and the diagnostic/monitoring method of the present invention are believed to be highly useful for the detection and monitoring of a variety of tumor types. Other diagnostic assays described prior to the present invention may rely on markers which are not necessarily present in all patients that have or are at risk of developing tumors (i.e., genetic markers that are predictive of only a subset of cancer patients, such as BRACI for breast cell tumors). Moreover, such markers are typically detected as an “all or nothing” response, and therefore provide only a “yes or no” answer and are not useful for staging tumors, for example. In contrast, the method of the present invention can be used for the detection of tumorigenicity or a potential therefore in any cell type that expresses FBLN-3, regardless of whether other genetic markers have predisposed an individual to the cancer. As discussed in the elsewhere herein, FBLN-3 is expressed in a large variety of tissue types. Moreover, the method of the present invention is designed to test for varying levels of FBLN-3 expression and/or biological activity as a marker of neoplastic transformation, as well as the form of FBLN-3 that is expressed by the cell (long or short), and therefore provides more than a “yes/no” answer in that tumor development in a patient can be staged using the test kit and method of the present invention. Therefore, the test kit and diagnostic method of the present invention are believed to be significantly more powerful and useful than previously described tumor assays.

According to the present invention, a means for detecting FBLN-3 expression or biological activity (including either form) can be any suitable reagent which can be used in a method for detection of FBLN-3 expression or biological activity as described previously herein. Such reagents include, but are not limited to: a probe that hybridizes under stringent hybridization conditions to a nucleic acid molecule encoding FBLN-3 or a fragment thereof, including to a FBLN-3-specific regulatory region in the FBLN-3-encoding gene (e.g., to a nucleic acid molecule comprising a nucleic acid sequence encoding any one of SEQ ID NOs:2, 3 or 4 or other FBLN-3 sequences); RT-PCR primers for amplification of mRNA encoding FBLN-3 or a fragment thereof (e.g., mRNA encoding any one of SEQ ID NOs:2, 3 or 4 or another FBLN-3 protein); and/or an antibody, antigen-binding fragment thereof or other antigen-binding peptide that selectively binds to FBLN-3 (e.g., any portion of SEQ ID NOs:2, 3, or 4 or another FBLN-3 protein, including portions that distinguish the long form from the short form, such as an epitope in the N-terminal 106 amino acids of the long form).

According to the present invention, a probe is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. As used herein, stringent hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

PCR primers are also nucleic acid sequences, although PCR primers are typically oligonucleotides of fairly short length that are used in polymerase chain reactions. PCR primers and hybridization probes can readily be developed and produced by those of skill in the art, using sequence information from the target sequence. (See, for example, Sambrook et al., supra or Glick et al., supra).

Antibodies that selectively bind to FBLN-3 in the sample can be produced using FBLN-3 protein information available in the art. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.). In one embodiment, antibodies that bind to an epitope (linear or conformational) of the long form, but not the short form, of FBLN-3 are envisioned. Such antibodies typically bind to an epitope that comprises at least a portion of the N-terminal 106 amino acids of long FBLN-3.

Antibodies useful in the test kit and methods of the present invention can include polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as antigen binding partners or antigen binding peptides, that have been designed to bind selectively to the protein of interest (FBLN-3). Examples of the design of such polypeptides, which possess a prescribed ligand specificity are given in Beste et al. (Proc. Natl. Acad. Sci. 96:1898-1903, 1999), incorporated herein by reference in its entirety.

The means for detecting a control marker characteristic of the cell type that is being sampled can generally be any type of reagent that can be used in a method of detecting the presence of a known marker in a sample, such as by a method for detecting the presence of FBLN-3 described previously herein. Specifically, the means is characterized in that it identifies a specific marker of the cell type being analyzed that positively identifies the cell type. For example, in a breast tumor assay, it is desirable to screen breast epithelial cells for the level of FBLN-3 expression and/or biological activity. Therefore, the means for detecting a control marker identifies a marker that is characteristic of an epithelial cell and preferably, a breast epithelial cell, so that the cell is distinguished from other cell types, such as a fibroblast. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The means for detecting of part (a) and or part (b) of the test kit of the present invention can be conjugated to a detectable tag or detectable label. Such a tag can be any suitable tag which allows for detection of the means of part (a) or (b) and includes, but is not limited to, any composition or label detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of part (a) and or part (b) of the test kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule. Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates. Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO₂, TiO₂, Al₂O₃, and NiO) and sand.

According to the present invention, the method and assay for assessing the tumorigenicity of cells in a patient, as well as other methods disclosed herein, are suitable for use in a patient that is a member of the Vertebrate class, Mammalia, including, without limitation, primates, livestock and domestic pets (e.g., a companion animal). Most typically, a patient will be a human patient. According to the present invention, the terms “patient”, “individual” and “subject” can be used interchangeably, and do not necessarily refer to an animal or person who is ill or sick (i.e., the terms can reference a healthy individual or an individual who is not experiencing any symptoms of a disease or condition).

Another embodiment of the present invention relates to a method to identify a compound useful for the inhibition of tumor cell growth or malignancy. Such a method includes the steps of: (a) detecting an initial level of FBLN-3 expression or activity in a tumor cell or soluble sample or a product derived from the tumor cell, and particularly a soluble product (e.g., cell supernate or lysate); (b) contacting the tumor cell with a test compound; (c) detecting a level of FBLN-3 expression or activity in the tumor cell (or sample derived therefrom) after contact of the tumor cell with the compound; and, (d) selecting a compound that changes the level of FBLN-3 expression or activity in the tumor cell, as compared to the initial level of FBLN-3 expression or activity, toward a baseline level of FBLN-3 expression or activity established from a non-tumor cell, wherein the selected compound is predicted to be useful for inhibition of tumor growth or malignancy. The method can include a further step of detecting whether a compound selected in (d) inhibits the growth or characteristics of malignancy (neoplastic transformation) of a tumor cell. The expected level of expression or activity of each form of FBLN-3 as they correlate with tumorigenicity has been discussed in detail above.

Steps (a) and (c) of the method of the present invention require detection of FBLN-3 expression and/or biological activity in a tumor cell or in a sample derived from the tumor cell, such as a cellular extract or supemate. Detection of FBLN-3 expression and/or biological activity can include, but is not limited to: detecting FBLN-3 mRNA transcription (e.g., by polymerase chain reaction (PCR), reverse transcriptase-PCR (RT-PCR), in situ hybridization, Northern blot, sequence analysis or detection of a reporter gene); detecting FBLN-3 translation (e.g., by immunoblot, enzyme-linked immunosorbant assay (ELISA), radioimmunoassay (RIA), immunoprecipitation, immunohistochemistry and immunofluorescence); and/or detecting FBLN-3 biological activity (e.g., by detecting any of the activities of FBLN-3 as described elsewhere herein or as known in the art). Such methods for detection of FBLN-3 expression and biological activity have been discussed in detail above with regard to the method for assessing tumorigenicity of cells, and that discussion applies to the detection FBLN-3 expression and biological activity here. The step of detection in step (a) is the control level of FBLN-3 expression or biological activity for a tumor-positive cell to which the detection in step (c) is to be compared and evaluated. The step of detection in step (c) is the experimental level of FBLN-3 expression or biological activity which indicates whether the test compound can change the level of FBLN-3 expression or biological activity in the cell, as compared to the level determined in step (a) and as compared to a baseline level of expression or activity that is established for a non-tumor cell. In other words, the assay determines whether a given compound is capable of changing FBLN-3 expression or activity from a tumor phenotype to or toward a non-tumor phenotype. The baseline level of FBLN-3 activity or expression established for a non-tumor cell can be determined by any of the methods as discussed previously herein for establishing a baseline level.

This reverse of this assay could also be performed using a normal, non-tumor cell (or sample derived therefrom), to identify compounds that change FBLN-3 expression or activity to or toward a tumor type. Such an assay could be a valuable assay to screen for putative carcinogens.

An assay similar to this assay can also be performed to detect FBLN-3 (long or short) and/or with other cell types (e.g., to detect the effect of a compound on the pro-angiogenic activities of long FBLN-3). Accordingly, another embodiment of the invention relates to a method to identify compounds that regulate the expression or biological activity of FBLN-3 (short or long) as potential anti-angiogenesis or pro-angiogenesis factors, comprising: (a) detecting an initial level of FBLN-3 expression or activity in a cell or soluble sample or product derived from the cell (e.g., cell supernate); (b) contacting the cell with a test compound; (c) detecting a level of FBLN-3 expression or activity in the cell (or sample derived therefrom) after contact of the cell with the compound; and, (d) selecting a compound that regulates the level of FBLN-3 expression or activity in the cell, as compared to prior to contact with the test compound. Typically, compounds that upregulate the expression or activity of long FBLN-3 in the presence of the compound can be selected as pro-angiogenic agents and wherein compounds that downregulate the expression or activity of long FBLN-3 in the presence of the compound can be selected as anti-angiogenic agents. The inverse is true when short FBLN-3 is the target. The method can include a further step of detecting whether a compound selected in (d) inhibits the pro-angiogenic capabilities of long FBLN-3 or enhances the anti-angiogenic capabilities of short FBLN-3, or vice versa. Therefore, the discussion regarding an assay described herein can readily be extended to these additional embodiments and is not limited to one particular form of FBLN-3 or tumor cells.

A cell suitable for use in the present method is any cell which expresses or can be induced to express, a detectable level of FBLN-3. A detectable level of FBLN-3 is a level which can be detected using any of the methods for FBLN-3 detection described herein. Since FBLN-3 is expressed by many mammalian cell types, a variety of cell types could be selected. However, it will be appreciated by those of skill in the art that some cell types are more suitable for use in an in vitro assay (e.g., easy to maintain in culture, easy to obtain), and that short FBLN-3 or long FBLN-3 may be more readily detectable in some cell types, and therefore, such cell types are preferable for use in the present invention. A preferred cell type to use in the method of the present invention is any cell type that has a high expression or low expression of short FBLN-3 in the tumor cell as compared to a non-tumor cell of the same cell type, so that a change in short FBLN-3 expression or activity is readily detectable. As discussed above, one can also use a sample derived from such a cell, such as a cell extract or cell supernate. Some preferred cells to use in the method of the present invention include, but are not limited to: fibroblasts (and fibrosarcomas), epithelial cells, and breast, colon, kidney, ovarian or uterine tumor cells and non-tumor cells that endogenously or recombinantly express long FBLN-3 for the assays for inhibitors of long FBLN-3. In one embodiment, a cell suitable for use in any aspect the general assay method is a cell which has been transfected with a recombinant nucleic acid molecule encoding FBLN-3 and operatively linked to a transcription control sequence so that FBLN-3 is expressed by the cell. Methods and reagents for preparing recombinant cells are known in the art.

As used herein, the term “putative regulatory compound” refers to compounds having an unknown or previously unappreciated regulatory activity in a particular process. The above-described method for identifying a compound of the present invention includes a step of contacting a test cell with a compound being tested for its ability to increase the expression or biological activity of short FBLN-3, in the case of the assay to identify compounds useful as cancer therapeutics or to decrease expression or biological activity of long FBLN-3, in the case of an assay to identify inhibitors of this pro-angiogenic factor. For example, test cells can be grown in liquid culture medium or grown on solid medium in which the liquid medium or the solid medium contains the compound to be tested. In addition, as described above, the liquid or solid medium contains components necessary for cell growth, such as assimilable carbon, nitrogen and micronutrients.

The above described methods, in one aspect, involve contacting cells with the compound being tested for a sufficient time to allow for interaction of the putative regulatory compound with an element that affects FBLN-3 expression and/or biological activity in a cell. Such elements can include, but are not limited to: a nucleic acid molecule encoding FBLN-3 (including regulatory regions of such a molecule), FBLN-3 protein, FBLN-3 inhibitors, FBLN-3 stimulators, and FBLN-3 substrates. The period of contact with the compound being tested can be varied depending on the result being measured, and can be determined by one of skill in the art. For example, for binding assays, a shorter time of contact with the compound being tested is typically suitable, than when activity or expression is assessed. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for the upregulation or downregulation of FBLN-3 expression or biological activity in a cell. It will be recognized that shorter incubation times are preferable because compounds can be more rapidly screened. A preferred incubation time is between about 1 hour to about 48 hours.

The conditions under which the cell or cell lysate of the present invention is contacted with a putative regulatory compound, such as by mixing, are any suitable culture or assay conditions and includes an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

In one aspect, the present method also makes use of non-cell based assay systems to identify compounds that can regulate FBLN-3 expression or biological activity and thereby are predicted to be useful for regulating cell growth. For example, FBLN-3 proteins and nucleic acid molecules encoding FBLN-3 may be recombinantly expressed and utilized in non-cell based assays to identify compounds that bind to the protein or nucleic acid molecule, respectively. In non-cell based assays the recombinantly expressed FBLN-3 or nucleic acid encoding FBLN-3 is attached to a solid substrate such as a test tube, microtiter well or a column, by means well known to those in the art.

In one embodiment, DNA encoding a reporter molecule can be linked to a regulatory element of the FBLN-3 gene (or a gene encoding a protein that directly regulates FBLN-3) and used in appropriate intact cells, cell extracts or lysates to identify compounds that modulate FBLN-3 gene expression, respectively. Appropriate cells or cell extracts are prepared from any cell type that normally expresses FBLN-3, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

Following steps (a), (b) and (c) of the present method is a step (d) of selecting a compound that changes the level of short FBLN-3 expression or activity in the tumor cell (or normal cell), as compared to the initial level of short FBLN-3 expression or activity, toward a baseline level of short FBLN-3 expression or activity established from a non-tumor cell (or tumor cell, if the test cell is a tumor cell). In other words, compounds which cause a change in the level of short FBLN-3 expression or biological activity in a tumor cell as detected in step (c) as compared to the level detected in step (a), toward the established baseline level for a non-tumor cell, are selected by the present method as being compounds that are predicted to be useful for the inhibition of carcinogenicity. Compounds that cause a change in the level of short FBLN-3 expression or biological activity in a non-tumor cell as detected in step (c) as compared to the level detected in step (a), toward the established baseline level for a tumor cell, are selected by the present method as being compounds that are predicted to be potential carcinogens.

Similarly, steps (a), (b) and (c) of the method to identify a compound that regulates long FBLN-3 include a step (d) of selecting a compound that regulates (up or down) the level of long FBLN-3 expression or activity in the cell, as compared to in the absence of the compound. Compounds which cause an increase in the level of long FBLN-3 expression or biological activity are selected by the present method as being compounds that are predicted to be useful as pro-angiogenesis agents. Compounds that cause a decrease in the level of long FBLN-3 expression or biological activity are selected by the present method as being compounds that are predicted to be useful as anti-angiogenesis agents (angiogenesis inhibitors).

Preferably, compounds which are selected in step (d) are compounds for which, after the test cell was contacted with the compound in step (b), the level of FBLN-3 expression or biological activity detected in step (c) was statistically significantly (i.e., with at least a 95% confidence level, or p<0.05) changed as compared to the initial level of short FBLN-3 expression or biological activity detected in step (a). Preferably, detection of at least about a 30% change in short FBLN-3 expression or biological activity in the cell as compared to initial level results in selection of the compound according to step (d). More preferably, detection of at least about a 50% change and more preferably at least about a 70% change, and more preferably at least about a 90% change, or any percentage change between 5% and higher in 1% increments (i.e., 5%, 6%, 7%, 8% . . . ) in FBLN-3 expression or biological activity in the cell as compared to the initial level results in selection of the compound according to step (d). In one embodiment, a 1.5 fold change in FBLN-3 expression or biological activity in the cell as compared to the initial level results in selection of the compound according to step (d). More preferably, detection of at least about a 3 fold change, and more preferably at least about a 6 fold change, and even more preferably, at least about a 12 fold change, and even more preferably, at least about a 24 fold change, or any fold change from 1.5 up in increments of 0.5 fold (i.e., 1.5, 2.0, 2.5, 3.0 . . . ) in FBLN-3 expression or biological activity as compared to the initial level, results in selection of the compound according to step (d).

It is to be understood that either of steps (a) and (c) of detection in any of the methods to identify a compound described above can result in no detection of FBLN-3 expression or biological activity or detection of FBLN-3. In addition, since the level of FBLN-3 expression or biological activity in step (a) (i.e., the initial level) is one of the baseline or control levels of FBLN-3 for the assay, if step (a) reveals no detectable FBLN-3 expression or biological activity, then any detectable level of FBLN-3 expression or biological activity in step (c) is considered to be a positive result and indicative of increased FBLN-3 activity in the cell and the appropriate assessment associated with this result. If the initial level of FBLN-3 expression or biological activity in step (a) is a detectable level, then the level of FBLN-3 expression or biological activity detected in step (c) is evaluated to determine whether it is statistically significantly greater than or less than that of step (a). It is possible that the level of FBLN-3 expression or biological activity in step (c) could be no detectable level, which would indicate that the compound did not increase or decrease FBLN-3 activity. In this scenario, however, it should be determined that the test cell can display an increase or decrease in FBLN-3 expression or biological activity under some conditions (i.e., by contact with a compound known to increase FBLN-3 activity in the test cell), so that false negatives are not identified.

In one embodiment of this method to identify regulators of short FBLN-3 of the present invention, the method further includes the step of detecting whether the compound selected in step (d) can inhibit tumor cell growth or a characteristic thereof. In this embodiment, the test cell is contacted with the compound as in step (b), and the growth characteristics of the cell before and after contact with the cell are evaluated. Evaluation of cell growth can be by any suitable method in the art, including, but not limited to, proliferation assays (e.g., by measuring uptake of [³H]-thymidine, viewing cells morphologically) and/or evaluating markers of cell growth (e.g., measurement of changes in cell surface markers, measurement of intracellular indicators of cell growth). Such methods are known in the art and are exemplified in the attached examples.

Compounds suitable for testing and use in the methods of the present invention include any known or available proteins, nucleic acid molecules, as well as products of drug design, including peptides, oligonucleotides, carbohydrates and/or synthetic organic molecules. Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety. Candidate compounds initially identified by drug design methods can be screened for the ability to modulate the expression and/or biological activity of FBLN-3 using the methods described herein.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands against a desired target, and then optimize the lead molecules by either random or directed design strategies. Methods of molecular diversity are described in detail in Maulik, et al., supra.

In a rational drug design procedure, the three-dimensional structure of a regulatory compound can be analyzed by, for example, nuclear magnetic resonance (NMR) or X-ray crystallography. This three-dimensional structure can then be used to predict structures of potential compounds, such as potential regulatory agents by, for example, computer modeling. The predicted compound structure can be used to optimize lead compounds derived, for example, by molecular diversity methods. In addition, the predicted compound structure can be produced by, for example, chemical synthesis, recombinant DNA technology, or by isolating a mimetope from a natural source (e.g., plants, animals, bacteria and fungi).

Various other methods of structure-based drug design are disclosed in Maulik et al., 1997, supra. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Compounds identified by the method described above can be used in a method to regulate cell growth, tumorigenicity, or angiogenesis, as described below and any such compounds are encompassed for use in the method described below.

Yet another embodiment of the invention relates to methods to increase or decrease the expression or biological activity of FBLN-3 (either form) in cells (e.g., isolated cells, cells of a tissue, cells in a patient) in order to achieve a goal. In the case of short FBLN-3, this goal can include, but is not limited to, reduction of angiogenesis in a tissue, decreased tumorigenicity of tumor cells, or reduction in the potential for development of tumor cells. Such a goal is primarily achieved by the upregulation (promotion of) short FBLN-3 expression or activity. However, to the extent that the expression of long FBLN-3 is determined to be associated with pro-angiogenesis in an undesirable setting (e.g. in a tumor cell), the method can include the inhibition of long FBLN-3. In the case of long FBLN-3, the method goal can include, but is not limited to, enhancement or promotion of angiogenesis in a tissue, or treatment of a disease or condition in which enhanced angiogenesis would be desirable. Such methods generally include the step of increasing or decreasing the expression and/or biological activity of FBLN-3, as required for a given cell type, in order to achieve the desired result (e.g., inhibition or promotion of angiogenesis, inhibition of tumorigenicity, etc.).

In the method of the present invention wherein the goals are to reduce angiogenesis in a tissue, decrease tumorigenicity of tumor cells or reduce the potential for the development of tumor cells, preferably, cells that are targeted by the method are cells which, prior to the application of the present method, are exhibiting inappropriate (malignant) cell growth or a potential therefore, or cells in a tissue where it is desirable to inhibit angiogenesis. Preferred cells to regulate according to this aspect of the present invention include tumor cells. Cells in which it is desirable to inhibit tumorigenicity or tissues in which inhibition of angiogenesis is desired can be identified, for example, using the method for assessing tumorigenicity or short FBLN-3 expression and activity of the present invention as described in detail above. Such methods are particularly useful in patients where increased tumorigenicity or angiogenesis is, or predicted to become, problematic. Therefore, such a method is particularly useful to treat patients that have, or are at a risk of developing, tumor cell growth (i.e., a cancer), or to treat any other patients having a condition characterized by undesirable cell growth (e.g., lymphoproliferative disorders). Other diseases and conditions in which inhibition of tumorigenicity or angiogenesis would be desirable will be apparent to those of skill in the art and are intended to be encompassed by the present invention.

Similarly, in the method of the present invention wherein the goals are to enhance or promote angiogenesis in a tissue, preferably, cells that are targeted by the method are cells in a tissue where it is desirable to promote angiogenesis. Preferred cells to regulate according to this aspect of the present invention include vascular endothelial cells. Such methods are particularly useful in patients where increased angiogenesis may be useful, such as in patients that have a vascular insufficiency or where the promotion of vascular stabilization and development is desired. Therefore, such a method is particularly useful to treat patients with vascular deficiencies, cardiovascular disease, or to stimulate endothelial cell activation and stabilization of newly formed microvessels or other vessels. Conditions in which promotion of angiogenesis would be desirable, including, but not limited to, stroke, ischemia, and similar conditions, will be apparent to those of skill in the art and are intended to be encompassed by the present invention.

The method of the present invention includes a step of modulating (i.e., upregulating or downregulating) FBLN-3 expression and/or biological activity in a patient that has, or is at risk of developing, inappropriate or unregulated cell growth or angiogenesis, or a patient or subject that is in need of promotion of angiogenesis, depending on the goal of the therapy, as discussed above. Modulating FBLN-3 expression or biological activity according to the present invention can be accomplished by directly affecting FBLN-3 expression (transcription or translation) or biological activity, or by directly affecting the ability of a regulator (inhibitor or stimulator) of FBLN-3 to bind to FBLN-3 or to activate FBLN-3. Preferably, the method of the present invention is targeted to a particular type of cell or tissue or region of the body in which inhibition of cell growth or regulation of angiogenesis is desired. A targeted cell, for example, could include a tumor cell, wherein the method does not substantially affect FBLN-3 expression or biological activity in non-tumor cells, or in cells of a different type that the tumor cell type. The targeted cell could also include endothelial cells, such as vascular endothelial cells. Therefore, the method of the present invention, in one embodiment, is intended to be specifically targeted to FBLN-3 expression and/or biological activity for the purpose of inhibiting or promoting cell growth, or inhibiting or promoting angiogenesis by modulating FBLN-3 expression and/or biological activity.

An increase in FBLN-3 (any form) expression and/or biological activity is defined herein as any measurable (detectable) increase (i.e., upregulation, stimulation, enhancement) of the expression or activity of FBLN-3. As used herein, to increase FBLN-3 expression and/or biological activity refers to any measurable increase in FBLN-3 expression and/or biological activity by any suitable method of measurement. A decrease in FBLN-3 (any form) expression and/or biological activity is defined herein as any measurable (detectable) decrease (i.e., downregulation, inhibition, reduction) of the expression or activity of FBLN-3. As used herein, to decrease FBLN-3 expression and/or biological activity refers to any measurable decrease in FBLN-3 expression and/or biological activity by any suitable method of measurement.

Accordingly, one embodiment of the present invention includes the use of a variety of agents (i.e., regulatory compounds) which, by acting directly on FBLN-3 (or the gene encoding FBLN-3) or on inhibitors or stimulators of FBLN-3, modulate (regulate up or down) the expression and/or biological activity of FBLN-3 in a cell to produce a desired effect (e.g., inhibition of tumorigenesis, inhibition or promotion of angiogenesis). Agents useful in the present invention include, for example, proteins, nucleic acid molecules, antibodies, and compounds that are products of rational drug design (i.e., drugs). Such compounds can be identified using the method of identifying compounds for regulating tumor cell growth and malignancy or for regulating angiogenesis as described above. Moreover, the expression or biological activity of FBLN-3 in a cell can be determined using the methods described above.

Therefore, in one embodiment, the method of the present invention increases the transcription and/or the translation of FBLN-3 by a cell in the patient that naturally expresses FBLN-3 and that is the target for growth regulation, or increases (stimulates, enhances) the biological activity of FBLN-3. Methods for increasing the expression of FBLN-3 include, but are not limited to, administering an agent that increases the expression or biological activity of endogenous FBLN-3 (either form), administering FBLN-3 protein or a homologue or analog (agonist) thereof to a subject, and/or overexpressing FBLN-3 in the target cells of the patient. In one aspect of this embodiment, FBLN-3 can be effectively overexpressed in a cell by increasing the activity of a FBLN-3 gene promoter in the cell such that expression of endogenous FBLN-3 in the cell is increased. For example, the activity of the FBLN-3 gene promoter can be increased by methods which include, contacting the promoter with a transcriptional activator, inhibiting a FBLN-3 inhibitor, and increasing the activity of a FBLN-3 stimulator. Methods by which such compounds (e.g., transcriptional activators) can be administered to a cell are described below. In another embodiment, FBLN-3 activity is increased by administering FBLN-3 or a homologue or analogue (synthetic homologue or mimetic) to the target cells or to the patient in an appropriate carrier or delivery vehicle.

In another embodiment, the method of the present invention decreases the transcription and/or the translation of FBLN-3 by a cell in the patient that naturally expresses FBLN-3 and that is the target for growth regulation, or inhibits the biological activity of FBLN-3. In this embodiment, it is desired to modify a target cell in order to decrease in FBLN-3 gene expression, decrease the function of the gene, or decrease the function of the gene product (i.e., the protein encoded by the gene). Such methods can be referred to as inactivation (complete or partial), deletion, interruption, blockage or down-regulation of a gene encoding FBLN-3. In one embodiment, reduction in FBLN-3 activity or expression is achieved by use of a FBLN-3 antagonist, which is any compound which inhibits (e.g., antagonizes, reduces, decreases, blocks, reverses, or alters) the effect of FBLN-3. Such antagonists can include, but are not limited to, a protein, peptide, or nucleic acid (including ribozymes, RNAi, aptamers and antisense) or product of drug/compound/peptide design or selection that provides the antagonistic effect.

As used herein, an anti-sense nucleic acid molecule is defined as an isolated nucleic acid molecule that reduces expression of a protein by hybridizing under high stringency conditions to a gene encoding the protein. Such a nucleic acid molecule is sufficiently similar to the gene encoding the protein that the molecule is capable of hybridizing under high stringency conditions to the coding or complementary strand of the gene or RNA encoding the natural protein. RNA interference (RNAi) is a process whereby double stranded RNA, and in mammalian systems, short interfering RNA (siRNA), is used to inhibit or silence expression of complementary genes. In the target cell, siRNA are unwound and associate with an RNA induced silencing complex (RISC), which is then guided to the mRNA sequences that are complementary to the siRNA, whereby the RISC cleaves the mRNA. A ribozyme is an RNA segment that functions by binding to the target RNA moiety and inactivate it by cleaving the phosphodiester backbone at a specific cutting site. A ribozyme can serve as a targeting delivery vehicle for a nucleic acid molecule, or alternatively, the ribozyme can target and bind to RNA encoding FBLN-3, for example, and thereby effectively inhibit the translation of FBLN-3. Aptamers are short strands of synthetic nucleic acids (usually RNA but also DNA) selected from randomized combinatorial nucleic acid libraries by virtue of their ability to bind to a predetermined specific target molecule with high affinity and specificity. Aptamers assume a defined three-dimensional structure and are capable of discriminating between compounds with very small differences in structure.

As used herein, reference to an isolated protein or polypeptide in the present invention, including an isolated FBLN-3 protein (either form), includes full-length proteins, fusion proteins, or any fragment or homologue of such a protein. The amino acid sequence for FBLN-3 from human, mouse and rat are described herein as exemplary FBLN-3 proteins. FBLN-3 Such a FBLN-3 protein can include, but is not limited to, purified FBLN-3 protein, recombinantly produced FBLN-3 protein, membrane bound FBLN-3 protein, FBLN-3 protein complexed with lipids, soluble FBLN-3 protein and isolated FBLN-3 protein associated with other proteins. More specifically, an isolated protein, such as a FBLN-3 protein, according to the present invention, is a protein (including a polypeptide or peptide) that has been removed from its natural milieu (i.e., that has been subject to human manipulation) and can include purified proteins,-partially purified proteins, recombinantly produced proteins, and synthetically produced proteins, for example. As such, “isolated” does not reflect the extent to which the protein has been purified. Preferably, an isolated FBLN-3 protein of the present invention is produced recombinantly. In addition, and by way of example, a “human FBLN-3 protein” refers to an FBLN-3 protein (generally including a homologue of a naturally occurring FBLN-3 protein) from a human (Homo sapiens) or to an FBLN-3 protein that has been otherwise produced from the knowledge of the structure (e.g., sequence) and perhaps the function of a naturally occurring FBLN-3 protein from Homo sapiens. In other words, a human FBLN-3 protein includes any FBLN-3 protein that has substantially similar structure and function of a naturally occurring FBLN-3 protein from Homo sapiens or that is a biologically active (i.e., has biological activity) homologue of a naturally occurring FBLN-3 protein from Homo sapiens as described in detail herein. As such, a human FBLN-3 protein can include purified, partially purified, recombinant, mutated/modified and synthetic proteins. According to the present invention, the terms “modification” and “mutation” can be used interchangeably, particularly with regard to the modifications/mutations to the amino acid sequence of FBLN-3 (or nucleic acid sequences) described herein. An isolated protein useful as an antagonist or agonist according to the present invention can be isolated from its natural source, produced recombinantly or produced synthetically.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

According to the present invention, an isolated FBLN-3 protein, including a biologically active homologue or fragment thereof, has at least one characteristic of biological activity of activity a wild-type, or naturally occurring FBLN-3 protein (which can vary depending on whether the homologue or fragment is an agonist, antagonist, or mimic of FBLN-3, and the isoform of FBLN-3). Biological activity of FBLN-3 and methods of determining the same have been described previously herein.

As used herein, the phrase “FBLN-3 agonist” refers to any compound that is characterized by the ability to agonize (e.g., stimulate, induce, increase, enhance, or mimic) the biological activity of a naturally occurring FBLN-3 as described herein, and includes any FBLN-3 homologue, binding protein (e.g., an antibody), agent that interacts with a protein or receptor bound by FBLN-3, or any suitable product of drug/compound/peptide design or selection which is characterized by its ability to agonize (e.g., stimulate, induce, increase, enhance) the biological activity of a naturally occurring FBLN-3 protein in a manner similar to the natural agonist, FBLN-3.

Similarly, the phrase, “FBLN-3 antagonist” refers to any compound which inhibits (e.g., antagonizes, reduces, decreases, blocks, reverses, or alters) the effect of an FBLN-3 agonist as described above. More particularly, a FBLN-3 antagonist is capable of acting in a manner relative to FBLN-3 activity, such that the biological activity of the natural agonist FBLN-3, is decreased in a manner that is antagonistic (e.g., against, a reversal of, contrary to) to the natural action of FBLN-3. Such antagonists can include, but are not limited to, a protein, peptide, or nucleic acid (including ribozymes, RNAi, aptamers, and antisense), antibodies and antigen binding fragments thereof, or product of drug/compound/peptide design or selection that provides the antagonistic effect.

Homologues of FBLN-3, including peptide and non-peptide agonists and antagonists of FBLN-3 (analogues), can be products of drug design or selection and can be produced using various methods known in the art. Such homologues can be referred to as mimetics. Mimetics have been described in detail above.

In one embodiment, a FBLN-3 homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring FBLN-3 amino acid sequence. A homologue of FBLN-3 differs from a reference (e.g., wild-type) FBLN-3 and therefore is less than 100% identical to the reference FBLN-3 at the amino acid level.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Sch\ddot{aa}ffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:
Reward for match=1
Penalty for mismatch=−2
Open gap (5) and extension gap (2) penalties
gap x_dropoff (50) expect (10) word size (11) filter (on)

For blastp, using 0 BLOSUM62 matrix:
Open gap (11) and extension gap (1) penalties
gap x_dropoff (50) expect (10) word size (3) filter (on).

In one aspect of this embodiment of the present invention, the expression and/or biological activity of FBLN-3 is increased by overexpressing FBLN-3 in the cell in which growth is to be regulated. Overexpression of FBLN-3 refers to an increase in expression of FBLN-3 over a normal, endogenous level of FBLN-3 expression. For some cell types, which do not express detectable levels of FBLN-3 under normal conditions, such expression can be any detectable level. For cell types which do express detectable levels of FBLN-3 under normal conditions, an overexpression is any statistically significant increase in expression of FBLN-3 (p<0.05) (or constitutive expression where expression is normally not constitutive) over endogenous levels of expression. One method by which FBLN-3 overexpression can be achieved is by transfecting the cell with a recombinant nucleic acid molecule encoding FBLN-3 operatively linked to a transcription control sequence, wherein the recombinant FBLN-3 is expressed by the cell. As discussed previously herein, the nucleic acid sequence encoding FBLN-3, vectors suitable for expressing such a molecule, and methods of transfection of a cell with such a molecule, including in vivo methods, are known and are described in detail below.

A recombinant nucleic acid molecule expressing FBLN-3 is a molecule that can include at least one of any nucleic acid sequence (e.g., SEQ ID NO:1) encoding a protein having FBLN-3 biological activity (e.g., SEQ ID NO:2, SEQ ID NO:3, SEQ ID NO:4 or a homologue thereof) operatively linked to at least one of any transcription control sequence capable of effectively regulating expression of the nucleic acid molecule(s) in the cell to be transfected. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

Preferably, a recombinant nucleic acid molecule is produced using recombinant DNA technology (e.g., polymerase chain reaction (PCR) amplification, cloning). Suitable nucleic acid sequences encoding FBLN-3 for use in a recombinant nucleic acid molecule of the present invention include any nucleic acid sequence that encodes a FBLN-3 having FBLN-3 biological activity and suitable for use in the target host cell. For example, when the target host cell is a human cell, human FBLN-3-encoding nucleic acid sequences are preferably used, although the present invention is not limited to strict use of naturally occurring sequences or same-species sequences.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes to screen appropriate libraries or DNA and PCR amplification of appropriate libraries or DNA using oligonucleotide primers. Preferred libraries to screen or from which to amplify nucleic acid molecule include mammalian genomic DNA libraries. Techniques to clone and amplify genes are disclosed, for example, in Sambrook et al., ibid.

A recombinant nucleic acid molecule includes a recombinant vector, which is any nucleic acid sequence, typically a heterologous sequence, which is operatively linked to the isolated nucleic acid molecule encoding a FBLN-3 protein, which is capable of enabling recombinant production of the FBLN-3 protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules. Recombinant vectors are preferably used in the expression of nucleic acid molecules, and can also be referred to as expression vectors. Preferred recombinant vectors are capable of being expressed in a transfected host cell, and particularly, in a transfected mammalian host cell in vivo.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more transcription control sequences. The phrase “operatively linked” refers to linking a nucleic acid molecule to a transcription control sequence in a manner such that the molecule is expressed when transfected (i.e., transformed, transduced or transfected) into a host cell.

Transcription control sequences are sequences that control the initiation, elongation, and termination of transcription. Particularly important transcription control sequences are those that control transcription initiation, such as promoter, enhancer, operator and repressor sequences. Suitable transcription control sequences include any transcription control sequence that can function in a host cell according to the present invention. A variety of suitable transcription control sequences are known to those skilled in the art. Preferred transcription control sequences include those which function in mammalian cells, with cell- or tissue-specific transcription control sequences being particularly preferred. Examples of preferred transcription control sequences include, but are not limited to, transcription control sequences useful for expression of a protein in breast epithelial cells and tumor cells and the naturally occurring FBLN-3 promoter. Particularly preferred transcription control sequences include inducible promoters, cell-specific promoters, tissue-specific promoters (e.g., insulin promoters) and enhancers. Suitable promoters for these and other cell types will be easily determined by those of skill in the art. Transcription control sequences of the present invention can also include naturally occurring transcription control sequences naturally associated with the protein to be expressed prior to isolation. In one embodiment, a transcription control sequence includes an inducible promoter.

One type of recombinant vector useful in a recombinant nucleic acid molecule of the present invention is a recombinant viral vector. Such a vector includes a recombinant nucleic acid sequence encoding a FBLN-3 protein of the present invention that is packaged in a viral coat that can be expressed in a host cell in an animal or ex vivo after administration. A number of recombinant viral vectors can be used, including, but not limited to, those based on alphaviruses, poxviruses, adenoviruses, herpesviruses, lentiviruses, adeno-associated viruses and retroviruses. Particularly preferred viral vectors are those based on adenoviruses and adeno-associated viruses. Viral vectors suitable for gene delivery are well known in the art and can be selected by the skilled artisan for use in the present invention. A detailed discussion of current viral vectors is provided in “Molecular Biotechnology,” Second Edition, by Glick and Pasternak, ASM Press, Washington D.C., 1998, pp. 555-590, the entirety of which is incorporated herein by reference.

For example, a retroviral vector, which is useful when it is desired to have a nucleic acid sequence inserted into the host genome for long term expression, can be packaged in the envelope protein of another virus so that it has the binding specificity and infection spectrum that are determined by the envelope protein (e.g., a pseudotyped virus). In addition, the envelope gene can be genetically engineered to include a DNA element that encodes and amino acid sequence that binds to a cell receptor to create a recombinant retrovirus that infects a specific cell type. Expression of the gene (i.e., the FBLN-3 gene) can be further controlled by the use of a cell or tissue-specific promoter. Retroviral vectors have been successfully used to transfect cells with a gene which is expressed and maintained in a variety of ex vivo systems.

An adenoviral vector infects a wide range of human cells and has been used extensively in live vaccines. Adenoviral vectors used in gene therapy do not integrate into the host genome, and therefore, gene therapy using this system requires periodic administration, although methods have been described which extend the expression time of adenoviral transferred genes, such as administration of antibodies directed against T cell receptors at the site of expression (Sawchuk et al., 1996, Hum. Gene. Ther. 7:499-506). The efficiency of adenovirus-mediated gene delivery can be enhanced by developing a virus that preferentially infects a particular target cell. For example, a gene for the attachment fibers of adenovirus can be engineered to include a DNA element that encodes a protein domain that binds to a cell-specific receptor. Examples of successful in vivo delivery of genes has been demonstrated and is discussed in more detail below.

Yet another type of viral vector is based on adeno-associated viruses, which are small, nonpathogenic, single-stranded human viruses. This virus can integrate into a specific site on chromosome 19. This virus can carry a cloned insert of about 4.5 kb, and has typically been successfully used to express proteins in vivo from 70 days to at least 5 months. Demonstrating that the art is quickly advancing in the area of gene therapy, however, a recent publication by Bennett et al. reported efficient and stable transgene expression by adeno-associated viral vector transfer in vivo for greater than 1 year (Bennett et al., 1999, Proc. Natl. Acad. Sci. USA 96:9920-9925).

Another type of viral vector that is suitable for use in the present invention is a herpes simplex virus vector. Herpes simplex virus type 1 infects and persists within nondividing neuronal cells, and is therefore a suitable vector for targeting and transfecting cells of the central and peripheral nervous system with a FBLN-3 protein of the present invention. Preclinical trials in experimental animal models with such a vector has demonstrated that the vector can deliver genes to cells of both the brain and peripheral nervous system that are expressed and maintained for long periods of time.

Suitable host cells to transfect with a recombinant nucleic acid molecule according to the present invention include any mammalian cell that can be transfected. Host cells can be either untransfected cells or cells that are already transfected with at least one nucleic acid molecule. Host cells according to the present invention can be any cell capable of producing a FBLN-3 protein as described herein or in which it is desired to produce FBLN-3.

According to the present invention, a host cell can also be referred to as a target cell or a targeted cell in vivo, in which a recombinant nucleic acid molecule encoding a FBLN-3 protein having FBLN-3 biological activity is to be expressed. As used herein, the term “target cell” or “targeted cell” refers to a cell to which a recombinant nucleic acid molecule of the present invention is selectively designed to be delivered. The term target cell does not necessarily restrict the delivery of a recombinant nucleic acid molecule only to the target cell and no other cell, but indicates that the delivery of the recombinant molecule, the expression of the recombinant molecule, or both, are specifically directed to a preselected host cell. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety). Ways in which viral vectors can be modified to deliver a nucleic acid molecule to a target cell have been discussed above. Alternatively, the route of administration, as discussed below, can be used to target a specific cell or tissue. For example, intracoronary administration of an adenoviral vector has been shown to be effective for the delivery of a gene cardiac myocytes (Maurice et al., 1999, J. Clin. Invest. 104:21-29). Intravenous delivery of cholesterol-containing cationic liposomes has been shown to preferentially target pulmonary tissues (Liu et al., Nature Biotechnology 15:167, 1997), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

According to the method of the present invention, a host cell is preferably transfected in vivo (i.e., in a mammal) as a result of administration to a mammal of a recombinant nucleic acid molecule, or ex vivo, by removing cells from a mammal and transfecting the cells with a recombinant nucleic acid molecule ex vivo. Transfection of a nucleic acid molecule into a host cell according to the present invention can be accomplished by any method by which a nucleic acid molecule administered into the cell in vivo, and includes, but is not limited to, transfection, electroporation, microinjection, lipofection, adsorption, viral infection, naked DNA injection and protoplast fusion. Methods of administration are discussed in detail below.

In one embodiment of the present invention, a recombinant nucleic acid molecule of the present invention is administered to a patient in a liposome delivery vehicle, whereby the nucleic acid sequence encoding the FBLN-3 protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a patient. A liposome delivery vehicle of the present invention comprises a lipid composition that is capable of fusing with the plasma membrane of the target cell to deliver the recombinant nucleic acid molecule into a cell.

A liposome delivery vehicle of the present invention can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands). Suitable liposomes for use with the present invention include any liposome. Preferred liposomes of the present invention include those liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art. According to the present invention, “extruded lipids” are lipids which are prepared similarly to MLV lipids, but which are subsequently extruded through filters of decreasing size, as described in Templeton et al., 1997, Nature Biotech., 15:647-652, which is incorporated herein by reference in its entirety. Small unilamellar vesicle (SUV) lipids can also be used in the composition and method of the present invention. In one embodiment, liposome delivery vehicles comprise liposomes having a polycationic lipid composition (i.e., cationic liposomes) and/or liposomes having a cholesterol backbone conjugated to polyethylene glycol. In a preferred embodiment, liposome delivery vehicles useful in the present invention comprise one or more lipids selected from the group of DOTMA, DOTAP, DOTIM, DDAB, and cholesterol.

According to the present invention, a regulatory compound for regulating (increasing or decreasing) the expression or biological activity of FBLN-3, including a recombinant nucleic acid molecule encoding FBLN-3, is typically administered to a patient in a composition. In addition to the recombinant nucleic acid molecule or other FBLN-3 regulatory compound (i.e., a protein, antibody, carbohydrate, small molecule product of drug design), the composition can include, for example, a pharmaceutically acceptable carrier, which includes pharmaceutically acceptable excipients and/or delivery vehicles, for delivering the recombinant nucleic acid molecule or other regulatory compound to a patient (e.g., a liposome delivery vehicle). As used herein, a pharmaceutically acceptable carrier refers to any substance suitable for delivering a therapeutic composition useful in the method of the present invention to a suitable in vivo or ex vivo site. Preferred pharmaceutically acceptable carriers are capable of maintaining a recombinant nucleic acid molecule of the present invention in a form that, upon arrival of the nucleic acid molecule to a target cell, the nucleic acid molecule is capable of entering the cell and being expressed by the cell. Suitable excipients of the present invention include excipients or formularies that transport or help transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into an animal. As used herein, a controlled release formulation comprises recombinant nucleic acid molecule or other FBLN-3 regulatory compound of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Suitable delivery vehicles have been previously described herein, and include, but are not limited to liposomes, viral vectors or other delivery vehicles, including ribozymes. Natural lipid-containing delivery vehicles include cells and cellular membranes. Artificial lipid-containing delivery vehicles include liposomes and micelles. As discussed above, a delivery vehicle of the present invention can be modified to target to a particular site in a patient, thereby targeting and making use of a nucleic acid molecule at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

As discussed above, a composition of the present invention is administered to a patient in a manner effective to deliver the recombinant nucleic acid molecule comprising a nucleic acid sequence encoding a FBLN-3 protein having FBLN-3 biological activity to a target cell, whereby the target cell is transfected by the recombinant molecule and whereby the FBLN-3 protein is expressed in the target cell. When another FBLN-3 regulatory compound is to be delivered to a target cell in a patient, the composition is administered in a manner effective to deliver the FBLN-3 regulatory compound to the target cell, whereby the compound can act on the cell (e.g., enter the cell and act on FBLN-3 or an inhibitor or stimulator thereof) so that FBLN-3 expression or biological activity is increased or decreased, depending on the isoform and the goal of the therapy. Suitable administration protocols include any in vivo or ex vivo administration protocol.

According to the present invention, an effective administration protocol (i.e., administering a composition of the present invention in an effective manner) comprises suitable dose parameters and modes of administration that result in transfection and expression of a recombinant nucleic acid molecule encoding a FBLN-3 protein or another FBLN-3 regulatory compound, in a target cell of a patient, and subsequent inhibition of the growth of the target cell or inhibition or promotion of angiogenesis, preferably so that the patient obtains some measurable, observable or perceived benefit from such administration. In some situations, where the target cell population is accessible for sampling, effective dose parameters can be determined using methods as described herein for assessment of tumor growth or using methods known in the art for the assessment of angiogenesis. Such methods include removing a sample of the target cell population from the patient prior to and after the recombinant nucleic acid molecule is administered, and measuring changes in FBLN-3 expression or biological activity, as well as measuring inhibition of the cell or impact on angiogenesis of a suitable cell line. Alternatively, effective dose parameters can be determined by experimentation using in vitro cell cultures, in vivo animal models, and eventually, clinical trials if the patient is human. Effective dose parameters can be determined using methods standard in the art for a particular disease or condition that the patient has or is at risk of developing. Such methods include, for example, determination of survival rates, side effects (i.e., toxicity) and progression or regression of disease.

According to the present invention, suitable methods of administering a composition of the present invention to a subject include any route of in vivo administration that is suitable for delivering the composition. The preferred routes of administration will be apparent to those of skill in the art, depending on the type of delivery vehicle used, the target cell population, whether the compound is a protein, nucleic acid, or other compound (e.g., a drug) and the disease or condition experienced by the patient. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor. For example, when the tumor is a breast tumor, the preferred methods of administration include impregnation of a catheter, and direct injection into the tumor.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a recombinant nucleic acid molecule to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. See, for example, U.S. Pat. No. 5,705,151; Aoki et al., 1997, J. Mol. Cell, Cardiol. 29:949-959; Kaneda et al., 1997, Ann N.Y Acad. Sci. 811:299-308; and von der Leyen et al., 1995, Proc Natl Acad Sci USA 92:1137-1141; Blaese et al., 1995, Science 270:475-480; Bordignon et al., 1995, Science 270:470-475; Koeberl et al., 1997, Proc Natl Acad Sci USA 94:1426-1431; Levine et al., 1998, J. Nutr. Sci. Vitaminol. 44:569-572; Millecamps et al., 1999, Nat. Biotechnol. 17:865-869; Oligino et al., 1999, Gene Ther. 6:1713-1720; Kuboki et al., 1999, Arch. Oral. Biol. 44:701-709; Apparailly et al., 1998, J. Immunol. 160:5213-5220; Ghivizzani et al., 1997, Gene Ther. 4:977-982; Evans and Robbins, 1996, Curr. Opin. Rheumatol. 8:230-234.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include a nucleic acid molecule of the present invention, and preferably includes a recombinant molecule of the present invention that preferably is replication, or otherwise amplification, competent. A naked nucleic acid reagent of the present invention can comprise one or more nucleic acid molecule of the present invention in the form of, for example, a dicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

In accordance with the present invention, a suitable single dose of a recombinant nucleic acid molecule encoding a FBLN-3 protein as described herein is a dose that is capable of transfecting a host cell and being expressed in the host cell at a level sufficient, in the absence of the addition of any other factors or other manipulation of the host cell, to inhibit the growth of the host cell when administered one or more times over a suitable time period. Doses can vary depending upon the cell type being targeted, the route of administration, the delivery vehicle used, and the disease or condition being treated.

In one embodiment, an appropriate single dose of a nucleic acid:liposome complex of the present invention is from about 0.1 μg to about 100 μg per kg body weight of the patient to which the complex is being administered. In another embodiment, an appropriate single dose is from about 1 μg to about 10 μg per kg body weight. In another embodiment, an appropriate single dose of nucleic acid:lipid complex is at least about 0.1 μg of nucleic acid, more preferably at least about 1 μg of nucleic acid, even more preferably at least about 10 μg of nucleic acid, even more preferably at least about 50 μg of nucleic acid, and even more preferably at least about 100 μg of nucleic acid.

Preferably, an appropriate single dose of a recombinant nucleic acid molecule encoding a FBLN-3 protein of the present invention results in at least about 1 pg of protein expressed per mg of total tissue protein per μg of nucleic acid delivered. More preferably, an appropriate single dose is a dose which results in at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

When the FBLN-3 regulatory agent is a protein, small molecule (i.e., the products of drug design) or antibody, a preferred single dose of such a compound typically comprises between about 0.01 microgram×kilogram⁻¹ and about 10 milligram×kilogram⁻¹ body weight of an animal. A more preferred single dose of an agent comprises between about 1 microgram×kilogram⁻¹ and about 10 milligram×kilogram⁻¹ body weight of an animal. An even more preferred single dose of an agent comprises between about 5 microgram×kilogram⁻¹ and about 7 milligram×kilogram⁻¹ body weight of an animal. An even more preferred single dose of an agent comprises between about 10 microgram×kilogram⁻¹ and about 5 milligram×kilogram⁻¹ body weight of an animal. Another particularly preferred single dose of an agent comprises between about 0.1 microgram×kilogram⁻¹ and about 10 microgram×kilogram⁻¹ body weight of an animal, if the agent is delivered parenterally.

In another embodiment, a targeting vector can be used to deliver a particular nucleic acid molecule into a recombinant host cell, wherein the nucleic acid molecule is used to delete or inactivate an endogenous gene (e.g., FBLN-3-encoding gene) within the host cell or microorganism (i.e., used for targeted gene disruption or knock-out technology). Such a vector may also be known in the art as a “knock-out” vector. In one aspect of this embodiment; a portion of the vector, but more typically, the nucleic acid molecule inserted into the vector (i.e., the insert), has a nucleic acid sequence that is homologous to a nucleic acid sequence of a target gene in the host cell (i.e., a gene which is targeted to be deleted or inactivated). The nucleic acid sequence of the vector insert is designed to bind to the target gene such that the target gene and the insert undergo homologous recombination, whereby the endogenous target gene is deleted, inactivated or attenuated (i.e., by at least a portion of the endogenous target gene being mutated or deleted).

Compositions of the present invention can be administered to any mammalian patient, and preferably to humans. According to one embodiment of the present invention, administration of a composition is useful to inhibit the tumorigenicity of a target cell or to inhibit angiogenesis in a tissue of a patient. Typically, it is desirable to inhibit the growth of a target cell (e.g., a tumor) to obtain a therapeutic benefit in the patient. In one embodiment, patients whom are suitable candidates for the method of the present invention include, but are not limited to, patients that have, or are at risk of developing (e.g., are predisposed to), cancer or a lymphoproliferative disease, or any condition in which regulation of angiogenesis might be beneficial. Particular conditions that are characterized or caused by abnormal or excessive angiogenesis, and therefore may be treated using the methods and compositions of the invention include, but are not limited to: cancer (e.g., activation of oncogenes, loss of tumor suppressors); infectious diseases (e.g., pathogens express angiogenic genes, enhance angiogenic programs); autoimmune disorders (e.g., activation of mast cells and other leukocytes); vascular malformations (e.g., Tie-2 mutation); DiGeorge syndrome (e.g., low VEGF and neuropilin-1 expression); HHT (e.g., mutations of endoglin or LK-1), cavernous hemangioma (e.g., loss of Cx37 and Cx40); atherosclerosis; transplant ateriopathy; obesity (e.g., angiogenesis induced by fatty diet, weight loss by angiogenesis inhibitors); psoriasis; warts; allergic dermatitis; scar keloids; pyogenic granulomas; blistering disease; Kaposi sarcoma in AIDS patients; persistent hyperplastic vitreous syndrome (e.g., loss of Ang-2 or VEGF164); diabetic retinopathy; retinopathy of prematurity; choroidal neovascularization (e.g., TIMP-3 mutation); primary pulmonary hypertension (e.g., germline BMPR-2 mutation, somatic EC mutation); asthma; nasal polyps; inflammatory bowel disease; periodontal disease; ascites; peritoneal adhesions; endometriosis; uterine bleeding; ovarian cysts; ovarian hyperstimulation; arthritis; synovitis; osteomyelitis; and osteophyte formation.

In another embodiment of the invention, patients whom are suitable candidates for a method of the invention include, but are not limited to: patients with vascular deficiencies, cardiovascular disease, or patients in whom stimulation of endothelial cell activation and stabilization of newly formed microvessels or other vessels would be beneficial. For example, such conditions include, but are not limited to, stroke, ischemia and related conditions.

Increasing or decreasing FBLN-3 expression or biological activity to inhibit tumorigenicity or to inhibit or promote angiogenesis in the absence of obtaining some therapeutic benefit is useful for the purposes of determining factors involved (or not involved) in a disease and preparing a patient to more beneficially receive another therapeutic composition. In a preferred embodiment, however, the methods of the present invention are directed to the inhibition of tumorigenicity of a target cell or inhibition or promotion of angiogenesis in a tissue, which is useful in providing some therapeutic benefit to a patient.

As such, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., metastatic tumor growth resulting from a primary cancer), and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). In particular, protecting a patient from a disease is accomplished by inhibiting the tumorigenicity of a target cell in the patient or inhibiting or promoting angiogenesis in the cells or tissues of a patient by regulating FBLN-3 expression or biological activity such that a beneficial effect is obtained. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

The following examples are provided for the purpose of illustration and are not intended to limit the scope of the present invention. Each publication or other reference disclosed below and elsewhere herein is incorporated herein by reference in its entirety.

### EXAMPLES

The following Materials and Methods were used in Examples 1-5.

Materials and Methods

Plasmids A retroviral vector encoding the short form of FBLN-3 (Lecka-Czernik et al., 1995) was constructed by PCR amplifying the full-length murine short FBLN-3 cDNA from EST BC031184. The resulting PCR fragment was ligated into pcDNA3.1/Myc-His B vector (InVitrogen, Carlsbad, Calif.) at Hind III (N-terminus) and Eco RI (C-terminus) restriction sites, which appended Mycand (His)6-tags to the C-terminus of FBLN-3. Afterward, the tagged FBLN-3 cDNA was PCR amplified using oligonucleotides containing Bgl II (N-terminus) and Xho I (C-terminus) restriction sites and ligated into identical sites in pMSCV-IRES-GFP (Schiemann et al., 2002). All FBLN-3 cDNA constructs were sequenced on an Applied Biosystems 377A DNA sequencing machine.

Cell Culture and Retroviral Infections Murine brain microvascular MB114 endothelial cells were cultured in Dulbecco's modified Eagle's medium (DMEM), 10% fetal bovine serum, 1× essential and nonessential amino acids, 50 μM β-mercaptoethanol, and 100 mM Hepes (pH 7.3) (see also Albig and Schiemann, 2004). Human dermal microvascular HMEC-1 endothelial cells were kindly provided by Dr. Gerard C. Blobe (Duke University), and were maintained in EBM-2 media supplemented with 2% FBS and growth factors according to the manufacture's recommendations (Cambrex, East Rutherford, N.J.).

Control (i.e., pMSCV-IRES-GFP) and FBLN-3 retroviral supernatants were produced by EcoPack2 retroviral packaging cells (Clontech, Palo Alto, Calif.) as described previously (Schiemann et al., 2002; Schiemann et al.; 2003), and subsequently were used to infect log-phase growing murine brain microvascular MB114 endothelial cells cultured in Dulbecco's modified Eagle's medium (DMEM), 10% fetal bovine serum, 1× essential and nonessential amino acids, 50 μM β-mercaptoethanol, and 100 mM Hepes (pH 7.3). Infected cells were isolated 48 h post-infection by FACS-sorting for GFP expression (highest 10%) to yield stable polyclonal populations of control or FBLN-3-expressing cells that were ≧90% positive for transgene expression. MB114 cells stably expressing FBLN-5 were described previously (U.S. patent application Publication No. 2004-0126788A1; Albig and Schiemann, 2004).

Fusion Protein Construction and Purification Glutathione S-transferase (GST) fusion proteins containing full-length murine short FBLN-3 or human FBLN-5 (less their signal sequences) were constructed by subcloning Eco RI/Not I- or Eco RI/Xho I-digested PCR fragments, respectively, into corresponding sites in pGEX-4T1 (Amersham Pharmacia Biotech). All fusion protein cDNA inserts were sequenced in their entirety on an Applied Biosystems 377A DNA sequencing machine. The expression and purification of various GST fusion proteins from transformed E. coli was performed as described previously (Sokol and Schiemann, 2004). All fusion protein preparations contained minute quantities of endotoxin, which was ≦0.2 ng/ml at fusion protein concentrations of 30 μg/ml.

Angiogenic Sprouting Assay Angiogenic sprouting of MB114 cells in collagen matrices was performed as described previously (U.S. patent application Publication No. 2004-0126788A1; Albig and Schiemann, 2004). Briefly, control- or FBLN-3-expressing MB114 cells (400,000 cells/well) were seeded onto 6-well plates containing 2 ml of solidified rat tail collagen, thus mimicking the activation of quiescent endothelial to initiate endothelial cell sprouting. In some experiments, control-, FBLN-3, or FBLN-5-expressing MB114 cells were allowed to tubulate in 3-dimensional rat tail collagen matrices in serum-free media supplemented with bFGF (300 ng/ml), thus enabling suspended endothelial cells to coalesce to into continuous networks that resemble capillary beds. Alternatively, MB114 cells were seeded onto collagen matrices in the absence or presence of increasing concentrations of recombinant GST or GSTFBLN-3 (0-50 μg/ml). Tubulation was allowed to proceed for 5 days, whereupon the number of invading angiogenic sprouts was quantified under a light microscope by determining the average number of sprouts present in 10 independent fields/well.

Endothelial Cell Activity Assays The effect FBLN-3 had on various MB114 cell activities was determined as follows: (i) cell proliferation using a [³H]thymidine incorporation as described (U.S. patent application Publication No. 2004-0126788A1; Schiemann, 2002; Albig and Schiemann, 2004); (ii) cell invasion using a modified Boyden-chamber assay and Matrigel matrices as described (U.S. patent application Publication No. 2004-0126788A1; Schiemann, 2002; Albig and Schiemann, 2004); (iii) cell migration using a modified Boyden-chamber assay and fibronectin (10 μg/ml) as described (U.S. patent application Publication No. 2004-0126788A1; Schiemann, 2002; Albig and Schiemann, 2004); and (iv) p38 MAPK activation using immunoblot analyses as described (U.S. patent application Publication No. 2004-0126788A1; Schiemann, 2002; Albig and Schiemann, 2004). In addition, the effect of recombinant FBLN-3, FBLN-5, or RGE-FBLN-5 on HMEC-1 endothelial cell adhesion was measured by coating 24-well plates overnight with 10 μg/ml of either fibronectin, GST, FBLN-3, FBLN-5, or RGE-FBLN-5. The following morning, the adhesive substrates were removed and the wells were subsequently blocked with 30 μg/ml of BSA for 60 min at room temperature. Afterward, HMEC-1 cells (100,000 cells/well) were allowed to adhere to the various substrates for 15 min, at which point nonadherent cells removed by two PBS washes. The remaining adherent cells fixed with 95% ethanol and stained with crystal violet, and subsequently quantified by extracting the crystal violet dye with 10% acetic acid, followed by spectrophotometry at 590 nm.

Alterations in MB114 cell MMP activity were determined by mixing control-, FBLN-3-, or FBLN-5-expressing MB114 cells (1.2×10⁶ cells/well) in 0.5 ml of collagen, which subsequently was allowed to solidify in 24-well plates. Twenty-four hours later, the media was discarded and the collagen matrices were removed and pelleted by microcentrifugation prior to fractionating supernatants (20-80 μl/lane) through 10% SDS-PAGE supplemented with 0.1% gelatin (Sigma, St. Louis, Mo.). Afterward, zymogram renaturing and developing were preformed using Novex Zymogram buffer system according to the manufacturer's instructions (InVitrogen, Carlsbad, Calif.).

Gene Expression Assays Total RNA from control-, FBLN-3-, and FBLN-5-expressing MB114 cells was purified using the RNAqueous kit, followed by an additional phenol/chloroform extraction and ethanol precipitation. cDNAs were synthesized by reverse transcribing total RNA (0.5 μg/reaction) with iScript reverse transcriptase according to the manufacturer's recommendations (BioRad, Hercules, Calif.). Semi-quantitative real-time PCR analyses were performed and analyzed on an ABI Prism 7000 Sequence Detection System (Applied Biosystems, Foster City, Calif.). Real-time PCR reactions (25 μl/reaction) used the SYBR green PCR system (Applied Biosystems) and contained 2.5 μl of 10-fold diluted cDNA and 0.1 μM of the following murine oligonucleotide pairs:

| | |
|---|---|
| (i) MMP-2, forward: | |
| 5′TAACCTGGATGCTGTCGTGGA; | (SEQ ID NO:6) |
| reverse: | |
| 5′GCCCAGCCAGTCTGATTTGAT; | (SEQ ID NO:7) |
| (ii) MMP-3, forward: | |
| 5′TGTTCCTGATGTTGGTGGCTT; | (SEQ ID NO:8) |
| reverse: | |
| 5′TGTCTTGGCAAATCCGGTG; | (SEQ ID NO:9) |
| (iii) TIMP-1, forward: | |
| 5′AAGCCTCTGTGGATATGCCCA; | (SEQ ID NO:10) |
| reverse: | |
| 5′AACCAAGAAGCTGCAGGCACT; | (SEQ ID NO:11) |
| (iv) TIMP-2, forward: | |
| 5′GTCCCATGATCCCTTGCTACA; | (SEQ ID NO:12) |
| reverse: | |
| 5′TGATGCAGGCGAAGAACTTG; | (SEQ ID NO:13) |
| (v) TIMP-3, forward: | |
| 5′CCCTGGCTATCAGTCCAAACA; | (SEQ ID NO:14) |
| reverse: | |
| 5′TGGCGTTGCTGATGCTCTT; | (SEQ ID NO:15) |
| (vi) TSP-1, forward: | |
| 5′GAACTCATTGGAGGTGCACGA; | (SEQ ID NO:16) |
| reverse: | |
| 5′TGGAACTTGTCATCCGGCA; | (SEQ ID NO:17) |
| and | |
| (vii) FBLN-3, forward: | |
| 5′GCAATGCTGGTGCTTGTGAA; | (SEQ ID NO:18) |
| reverse: | |
| 5′ACAGAGCTTGTGCGGAAGGTT. | (SEQ ID NO:19) |

Relative gene expression levels were determined according to the manufacturer's recommendations, and subsequently normalized to corresponding GAPDH signals.

In Vivo Angiogenesis Assay The effect of FBLNs 3 and 5 on angiogenesis and vessel infiltration in vivo was determined using the Matrigel implantation assay. Briefly, 6 wk old C57BL/6 female mice were injected twice subcutaneously in the ventral groin area with Matrigel (700 μl/injection; BD Biosciences, Bedford, Mass.) supplemented either with diluent (PBS), or with bovine bFGF (300 ng/ml; R&D Systems, Minneapolis, Minn.) together with either recombinant GST (50 μg/ml), FBLN-3 (10 or 50 μg/ml), FBLN-5 (10 or 50 μg/ml), or RGE-FBLN-5 (10 or 50 μg/ml). Seven days post-implantation, the mice were sacrificed and the plugs harvested. For each mouse, one plug was fixed overnight in 10% formalin prior to embedding in paraffin and sectioning in the National Jewish Histology Laboratory. Afterward, the sections were stained using the Masson's trichrome procedure to visualize infiltrating vessels, which were quantified under a light microscope by determining the average number of vessels present in ≧10 independent fields on two independent slides. Only fields containing at least one vessel in the area underlying the skin were quantified. The second plug was weighed and homogenized in PBS prior to determining plug hemoglobin content by Drabkin's assay according to the manufacturer's recommendations (Sigma, St. Louis, Mo.). Each sample was assayed in duplicate and resulting hemoglobin contents were normalized to corresponding plug protein concentrations. Two mice were used per experimental condition and this experiment was performed three times in its entirety. All animal studies were performed according to protocol procedures approved by the Animal Care and Use Committee at National Jewish Medical and Research Center.

Tumor Studies Murine MCA102 fibrosarcoma cells (Routes et al., 2005; Wexler and Rosenberg, 1979) were kindly provided by Dr. John M. Routes (National Jewish Medical and Research Center) and were engineered by retroviral transduction to stably express either GFP, FBLN-3, or FBLN-5. To maximize FBLN-3 secretion from MCA102 cells, the inventors first shuttled the murine short FBLN-3 cDNA (less its signal sequence) through the pSecTag vector (InVitrogen) at Hind III (N-terminus) and Not I (Cterminus) restriction sites. In addition to C-terminally tagging the FBLN-3 cDNA with the Mycand (His)₆-tags, the pSecTag vector also appended the Igκ leader sequence to its N-terminus, thus permitting more efficient FBLN-3 secretion when introduced into MCA102 cells (data not shown). Afterward, the resulting tagged FBLN-3 cDNA fragment was PCR amplified using oligonucleotides containing Bgl II (N-terminus) and Eco RI (C-terminus) restriction sites, and subsequently ligated into identical sites in pMSCV-IRES-GFP to facilitate retroviral production. The effect of FBLNs 3 and 5 on tumor growth and angiogenesis was measured by subcutaneously injecting 10 wk old C57BL/6 female mice with GFP-, FBLN-3-, or FBLN-5-expressing MCA102 fibrosarcoma cells (500,000 cells/implant). Twenty-one days postimplantation, the mice were sacrificed and the tumors were harvested, weighed, and fixed overnight in 10% formalin prior to sectioning and H&E staining in the National Jewish Histology Laboratory. Afterward, tumor blood vessel densities were quantified under a light microscope by determining the average number of vessels present in 5 independent fields per slide. As for Matrigel implantation assays, only those fields containing at least one vessel were quantified. Six mice were used per experimental condition and this experiment was performed three times in its entirety.

Statistical Analyses Data were analyzed using Student's t-test and all comparisons for in vitro studies were made to GFP-expressing MB114 cells, while those for in vivo studies were made to bFGF-supplemented Matrigel plugs. A value of P<0.05 was considered significant.

The data shown in Examples 1-5 below are directed to the short form of FBLN-3 (i.e., lacking the N-terminal 106 amino acids as compared to the long form of FBLN-3).

### Example 1

The following example shows that FBLN-3 is homologous to FBLN-5 and is expressed aberrantly during tumorigenesis.

Murine FBLN-3 (short form) is 48% identical to murine FBLN-5 (FIG. 1A), and like FBLN-5 (Schiemann et al., 2002; Nakamura et al., 1999; Kowal et al., 1999), is widely expressed throughout mammalian tissues (FIG. 1B; Ikegawa et al., 1996; Giltay et al., 1999) and regulates cell proliferation in a context-specific manner (Lecka-Czernik et al., 1995). FIG. 1B shows phosphor images visualized using radiolabeled cDNA probes corresponding to either murine FBLN-3 (left panel) or human ubiquitin (middle panel) that were hybridized to a matched human normal:tumor cDNA array. FIG. 1B shows FBLN-3 and ubiquitin expression in paired normal (upper spot) and malignant (bottom spot) tissues. FBLN-3 expression was normalized to that of ubiquitin, followed by a determination of tumor:normal tissue FBLN-3 expression ratios. Ratios ≧2 or≦0.5 were considered significant. Tumor type and metastasis status are indicated by: (i) open boxes, no information; (ii) filled boxes, metastasis observed; and (iii) stripped boxes, metastasis not observed (right panel) (K, kidney. S.I., small intestine).

Also analogous to FBLN-5, the present inventors find that tumorigenesis negatively impacts tissue expression of FBLN-3. Indeed, FBLN-3 expression was altered aberrantly in 47% ( 32/68) of sampled tumors, of which 78% ( 25/32) exhibited downregulation and 22% ( 7/32) upregulation of FBLN-3 mRNA (FIG. 1B). Reduced FBLN-3 expression was especially evident in cancers of the breast (67%; 6/9cases), ovary (67%; ⅔ cases), lung (67%; ⅔ cases), kidney (47%; 7/15 cases), and colon (46%; 5/11 cases). Collectively, these findings establish tumorigenesis as a negative regulator of FBLN-3 expression within tumor microenvironments, and indicate that FBLN-3, like FBLN-5, functions in suppressing tumor formation and progression in humans.

### Example 2

The following example demonstrates that short FBLN-3 antagonizes angiogenic activities and sprouting in endothelial cells.

Because tumorigenesis downregulates the expression of FBLNs 3 and 5 (see above), and because these ECM proteins have similar structures and expression profiles, the present inventors hypothesized that FBLN-3 may be functionally similar with FBLN-5 in mediating anti-angiogenic activities. Tubulating MB114 cells were incubated in collagen matrices for varying times as indicated in FIG. 2A. Total RNA was isolated and used to measure FBLN-3 expression by quantitative real-time PCR. The resulting GFP expression profiles in MB114 cells infected with control (FIG. 2B; left panel) or FBLN-3- (FIG. 2B; right panel) retroviruses were also evaluated. In these experiments, conditioned-media from control- and FBLN-3-expressing MB114 cells was tumbled with Ni++-agarose and captured protein complexes were immunoblotted with anti-Myc antibodies to visualize recombinant FBLN-3 protein. As shown in FIG. 2C, the invasion (white bar; n=2), proliferation (black bar; n=3), and angiogenic sprouting (gray bar; n=5) of control- and FBLN-3-expressing MB114 cells were compared (data are mean±SE. *, P<0.05).

Interestingly, whereas tubulogenesis downregulates FBLN-5 expression (U.S. patent application publication No. 2004-0126788A1; Albig and Schiemann, 2004), FBLN-3 mRNA expression was upregulated significantly in tubulating MB114 cells (FIG. 2A; Data are mean±SE (n=4). *, P<0.05). However, analogous to FBLN-5 (U.S. patent application Publication No. 2004-0126788A1; Albig and Schiemann, 2004), constitutive FBLN-3 expression (FIG. 2B) significantly inhibited MB114 cell proliferation and invasion (FIG. 2C), which are critical processes during angiogenesis activation and endothelial cell sprouting. Accordingly, FBLN-3 expression significantly reduced angiogenic sprouting by quiescent layers of MB114 cells (FIG. 2C). Moreover, administering increasing concentrations of recombinant FBLN-3 to MB114 cells dose-dependently antagonized their angiogenic sprouting, with half-maximal inhibition (IC₅₀) occurring at ˜6 μg/ml (FIG. 2D). In the experiment shown in FIG. 2D, MB114 cell tubulation proceeded on collagen gels for 5 days in the absence or presence of increasing concentrations of recombinant FBLN-3 (0-50 μg/ml). The quantity of invading angiogenic sprouts was determined by manual counting under a light microscope (data are the mean±SD from a single experiment that was repeated once with similar results). Interestingly, FBLN-5 reduced endothelial cell response to VEGF in part by preventing VEGF stimulation of ERK1/ERK2 and p38 MAPK (U.S. patent application Publication No. 2004-0126788A1; Albig and Schiemann, 2004).

Control- or FBLN-3-expressing MB114 cells were stimulated with VEGF165 (50 ng/ml) for 0-60 min. Whole cell extracts were immunoblotted with phospho-ERK1/ERK2 and -p38 MAPK antibodies, and subsequently reprobed with anti-ERK1 and -p38 MAPK antibodies to monitor differences in protein loading. Data are shown in FIG. 2E as the mean±SE (n=2) phosphorylation of ERK1/ERK2 or p38 MAPK stimulated by VEGF (*, P<0.05). FIG. 2E shows that FBLN-3 expression had little affect on VEGF165 stimulation of ERK1/ERK2, but significantly inhibited that of p38 MAPK (#, P <0.05) in MB114 cells. Taken together, these findings establish FBLN-3 as a novel antagonist of endothelial cell activities coupled to angiogenic sprouting, as well as demonstrate the overlapping anti-angiogenic activities possessed by FBLNs 3 and 5.

### Example 3

The following examples shows altered endothelial cell expression of MMPs, TIMPs, and TSP-1 by FBLNs 3 (short form) and 5.

The inhibition of endothelial cell invasion and angiogenic sprouting by FBLNs 3 and 5 implicated these proteins as potential regulators of the expression and activity of ECM proteases, particularly MMPs and their inhibitors, the TIMPs. The present inventors tested this hypothesis by performing semi-quantitative real-time PCR analyses on total RNA isolated from control-, FBLN-3-, and FBLN-5-expressing MB114 cells. Briefly, total RNA isolated from resting FBLN-5 (FIG. 3A; filled bars)- and FBLN-3 (FIG. 3A; open bars)-expressing MB114 cells was reverse transcribed prior to analyzing the expression of MMP-2, MMP-3, TIMP-1, TIMP-2, TIMP-3, and TSP-1 by quantitative real-time PCR (data are the mean±SE (n=3)). FIG. 3A shows that relative to control cells, FBLNs 3 and 5 both decreased MB114 cell expression of MMP-2 and MMP-3, while simultaneously increasing that of the MMP antagonists, TIMP-1 and TIMP-3. Although MB114 cell expression of TIMP-2 was unaffected by either FBLN-3 or FBLN-5, both FBLNs induced MB114 cell expression of the anti-angiogenic molecule, TSP-1 (FIG. 3A).

More importantly, both FBLNs also inhibited MMP-2 and -3 transcript synthesis in tubulating MB114 cells suspended in 3-dimensional rat tail collagen matrices (FIG. 3B). In this experiment, control (FIG. 3B; filled squares), FBLN-3 (FIG. 3B; open squares)-, and FBLN-5 (open circles)-expressing MB114 cells were allowed to tubulate in collagen gels for 0-24 h. Total RNA was isolated and subjected to semi-quantitative PCR to monitor MMP-2, MMP-3, and TIMP-3 expression (data are the mean±SE (n=4)). Interestingly, although TIMP-3 expression was elevated basally in FBLN-5-expressing MB114 cells, only FBLN-3 stimulated TIMP-3 expression in tubulating MB114 cells (FIG. 3B).

Gelatin zymography of MB114 cell conditioned-media confirmed that expression of either FBLN-3 or FBLN-5 significantly reduced MMP-2 protease activity in tubulating MB114 cells (FIG. 3C). Briefly, control, FBLN-5-, and FBLN-3-expressing MB114 cells were allowed to tubulate in collagen gels for 24 h, and the resulting conditionedmedia was fractionated through 10% SDS-PAGE supplemented with 0.1% gelatin. Afterward, the gel was processed for gelatin zymography (data are the mean±SE (n=3). BG, background buffer control. *, P<0.05). Collectively, these findings indicate that FBLNs 3 and 5 promote angiogenesis resolution by targeting MMP, TIMP, and TSP-1 expression in resting (i.e., steady-state) and activated endothelial cells, thereby reducing ECM proteolysis and remodeling.

### Example 4

The following example shows that FBLNs 3 (short form) and 5 antagonize angiogenesis in vivo.

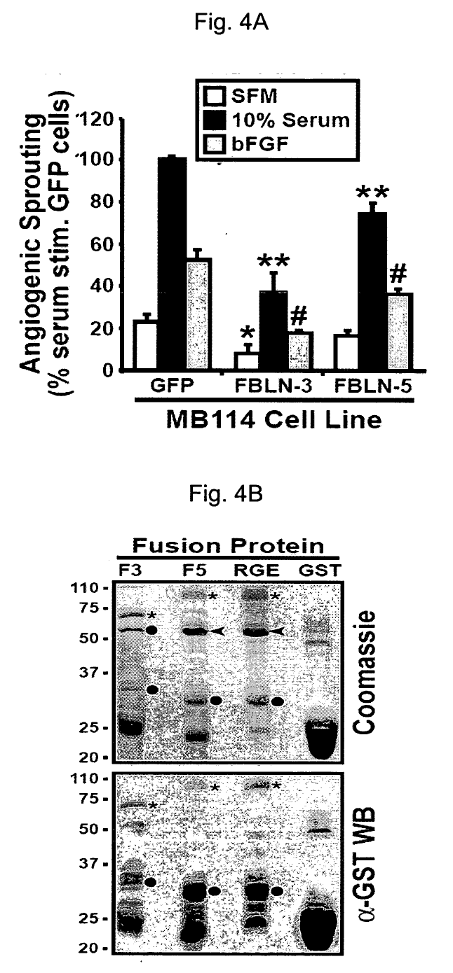

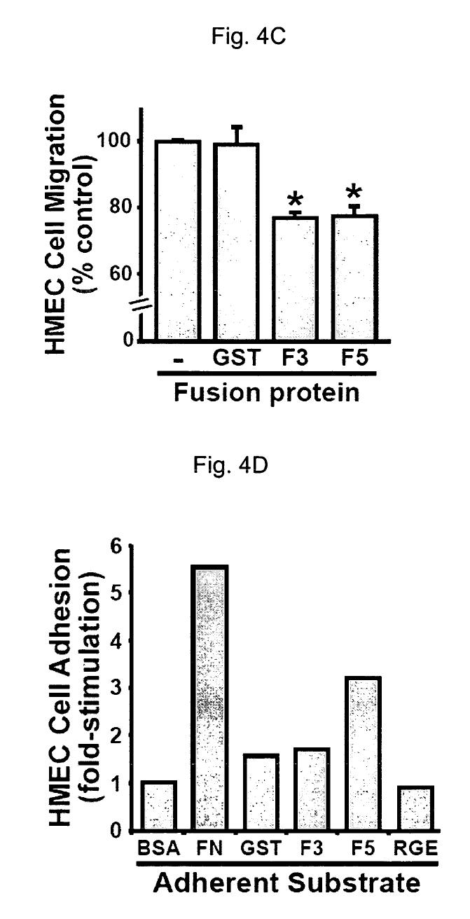

The present inventors' previous FBLN-5 findings (U.S. patent application Publication No. 2004-0126788A1; Albig and Schiemann, 2004) and those presented herein with regard to FBLN-3 together identify FBLNs 3 and 5 as novel inhibitors of endothelial cell activities and angiogenic sprouting in vitro. The present inventors next asked whether these FBLNs could prevent new vessel growth in vivo. In doing so, the present inventors monitored vessel development and infiltration into Matrigel plugs that were supplemented with pro-angiogenic factors and implanted subcutaneously into mice. Preliminary experiments established that bFGF was significantly better than VEGF in mediating vessel development in Matrigel plugs (data not shown). Angiogenic sprouting of control-, FBLN-3-, or FBLN-5-expressing MB114 cells was allowed to proceed for 5 days in collagen matrices incubated in complete media (FIG. 4A; 10% serum; black bars), or in serum-free media (FIG. 4A; SFM; white bars) supplemented with 300 ng/ml bFGF (FIG. 4A; bFGF; gray bars). The quantity of invading angiogenic sprouts was determined by manual counting under a light microscope. Interestingly, similar to their inhibitory effects on VEGF signaling in endothelial cells, FBLNs 3 and 5 both significantly antagonized bFGF stimulation of MB114 cell angiogenic sprouting in vitro (FIG. 4A; data are the mean±SE (n=4). *, **, and #, P<0.05).

Moreover, recombinant FBLNs 3 and 5 (FIG. 4B) both inhibited human dermal microvascular HMEC-1 endothelial cell migration to fibronectin (FIG. 4C), while FBLN-5, but not FBLN-3, mediated their adhesion in an RGD-dependent manner (FIG. 4D; Nakamura et al., 1999; Nakamura et al., 2002). In these experiments, recombinant FBLN-3 (F3), FBLN-5 (F5), RGE-FBLN-5 (RGE), or GST were purified from detergent-solubilized bacterial lysates by glutathione affinity chromatography. Shown in FIG. 4B is the purity of recombinant FBLNs (2 μg) monitored by coomassie staining, or by immunoblotting with anti-GST antibodies as indicated (asterisks show full-length GST-FBLN fusion proteins, while circles indicate major GST-immunoreactive FBLN cleavage products; arrowheads represent FBLN-5 immunoreactive products (data not shown)). HMEC-1 cells were allowed to migrate to fibronectin in the absence or presence of 10 μg/ml of recombinant GST, FBLN-3, or FBLN-5 as indicated in FIG. 4C (data are the mean±SE (n=3). *, P<0.05). Finally, HMEC-1 cells were allowed to adhere 10 μg/ml of either fibronectin (FN), GST, FBLN-3 (F3), FBLN-5 (F5), or RGE-FBLN-5 (RGE) for 15 min at 37° C. Data in FIG. 4D depict the foldstimulations of cell adhesion normalized to BSA coated wells from a representative experiment that was repeated twice with similar results. Collectively, these findings show that the angiostatic effects of FBLNs 3 and 5 are not restricted solely to murine endothelial cells, and more importantly, that the angiostatic activities of these FBLNs are not targeted solely to VEGF signaling in endothelial cells.

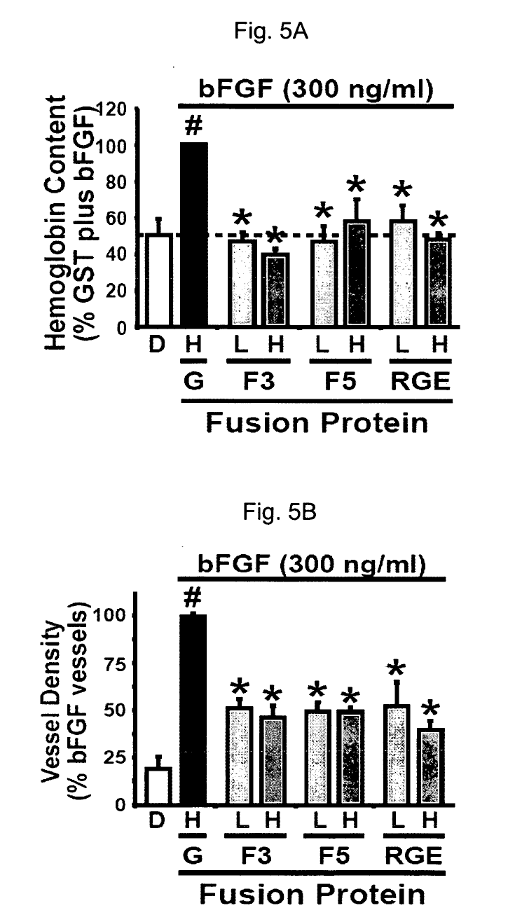

In a further experiment, C57BL/6 female mice were injected subcutaneously with Matrigel supplemented with: diluent (D), bFGF (300 ng/ml) in presence of 50 μg/ml (H) recombinant GST, or bFGF (300 ng/ml) in the presence of either 10 μg/ml (L) or 50 μg/ml (H) of recombinant FBLN-3 (F3), FBLN-5 (F5), or RGE-FBLN-5 (RGE) as indicated in FIGS. 5A and 5D. Mice were sacrificed on day 7 and the plugs harvested and prepared for angiogenesis analyses. FIG. 5A shows hemoglobin contents determined by Drabkin's assay corresponding to representative vascularization (data not shown) observed in harvested Matrigel plugs (data are the mean±SE (n=3). *, P<0.05.) FIG. 5B shows vessel densities determined by manual counting corresponding to representative infiltrating vessels (data not shown) in Masson's trichrome-stained Matrigel sections (data in FIG. 5B are the mean±SE (n=3). *, P<0.05).

The results showed that bFGF stimulated significant vascularization of implanted Matrigel plugs. The extent of Matrigel vascularization was quantified by measuring plug hemoglobin contents (FIG. 5A) and microvessel densities (FIG. 5B). The present inventors have found FBLN-5 to be a potent inducer of fibroblast invasion in vitro in part through its ability to alter fibroblast ECM transcriptome profiles (data not shown). Accordingly, FBLNs 3 and 5 both stimulated significantly more fibroblast invasion into Matrigel plugs as compared to bFGF (FIG. 5B). However, despite their fibroblast activating activities and consistent with their endothelial inhibiting activities, both FBLNs prevented bFGF stimulation of vessel development and angiogenesis in vivo (FIGS. 5A and 5B). In accordance with the present inventors' previous study (U.S. patent application Publication No. 2004-0126788A1; Albig and Schiemann, 2004), the in vivo anti-angiogenic activities of FBLN-5 were independent of its integrin-binding RGD motif (FIGS. 5A and 5B). Taken together, these findings demonstrate that FBLNs 3 and 5 are inhibitors of angiogenesis in vivo, and as such, are expected to also be inhibitors of tumorigenesis.

### Example 5

The following example demonstrates that short FBLN-3 and FBLN-5 antagonize the growth and vascularization of tumors in mice.

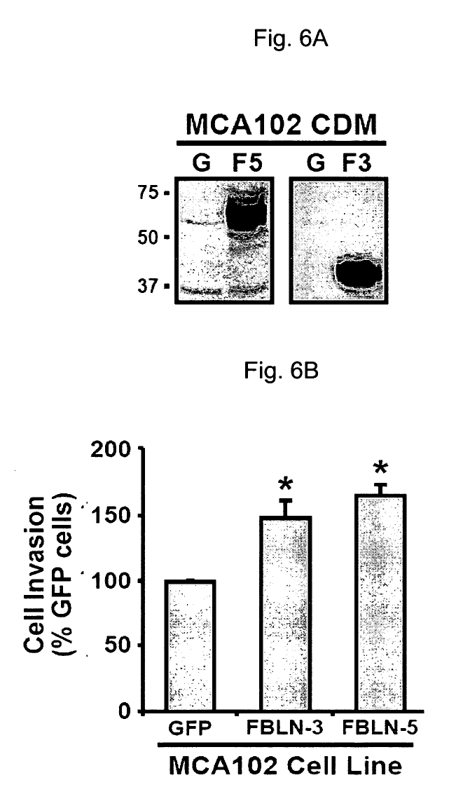

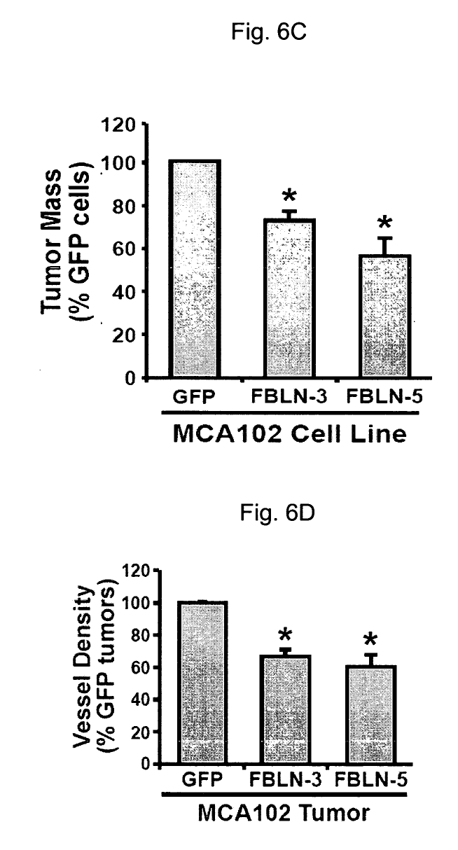

The present inventors' findings thus far show that neovascularization is inhibited by FBLNs 3 and 5 in vivo (FIGS. 5A and 5B): they also suggest that tumor growth and angiogenesis may be reduced by augmenting the concentration of either FBLN within tumor microenvironments. To test this hypothesis, MCA102 fibrosarcoma cells were engineered by retroviral transduction to stably express murine versions of either FBLN-3 or FBLN-5 (FIG. 6A). Briefly, conditioned-media from GFP (G)-, FBLN-5 (F5)-, or FBLN-3 (F3)-expressing MCA102 fibrosarcoma cells was tumbled with Ni++-agarose and captured protein complexes were immunoblotted with anti-Myc antibodies to visualize recombinant FBLN proteins. The present inventors chose to study MCA102 fibrosarcoma cells for two reasons. First, MCA102 fibrosarcoma cells were created by 3-methylcholanthrene treatment of C57BL/6 mice (Wexler and Rosenberg, 1979), thus enabling the inventors to monitor the effects of FBLNs 3 and 5 on tumor growth and angiogenesis in genetically normal mice. And second, the present inventors and colleagues showed previously that FBLN-5 inhibits the proliferation of mink lung epithelial cells, while augmenting the proliferation, migration, and invasion of human HT1080 fibrosarcoma cells in vitro (Schiemann et al., 2002), indicating that FBLN-5 regulates various cellular responses in a context-specific manner. Thus, MCA102 fibrosarcoma cells afforded a unique opportunity to study the context-specific effects of FBLNs 3 and 5 on cancer cell activities in vitro, and on cancer cell behavior in vivo.

As expected, MCA102 fibrosarcoma cells engineered to constitutively express FBLN-5 synthesized more DNA (15±1.3%; n=4; P=0.0014) than did their control counterparts, Somewhat surprisingly, FBLN-3 expression had no effect on MCA102 fibrosarcoma proliferation (data not shown), while expression of both FBLNs significantly enhanced MCA102 fibrosarcoma invasion through synthetic basement membranes (FIG. 6B; data are the mean±SE (n=4). *, P<0.05). Thus, similar to the present inventors' previous findings describing the oncogenic activities of FBLN-5 in human fibrosarcoma cells (U.S. patent application Publication No. 2004-0126788A1; Schiemann et al., 2002), these findings show that FBLN-3 and FBLN-5 both increased the apparent tumorigenic potential of MCA102 fibrosarcoma cells when assayed in vitro. Moreover, these results suggest that the growth and angiogenesis of MCA102 tumors in mice would be enhanced by the expression of FBLNs 3 and 5.

In stark contrast, both FBLNs significantly reduced the growth and mass of MCA102 tumors produced in syngeneic C57BL/6 mice (FIG. 6C). In this experiment, C57BL/6 female mice were injected subcutaneously with control (GFP)-, FBLN-3-, or FBLN-5-expressing MCA102 fibrosarcoma cells. Mice were sacrificed on day 21, and the resulting tumors were removed and weighed (FIG. 6C; mean (±SE; n=3) tumor weights are reported as the percent GFP tumor weight. *, P<0.05).

More importantly, tumors derived from FBLN-3- or FBLN-5-expressing MCA102 fibrosarcoma cells also exhibited significantly reduced blood vessel densities as compared to tumors derived from control cells (FIG. 6D). In addition, FBLN-3-expressing MCA102 tumors contained significantly enlarged (1.85±0.18-fold; n=3; P=0.04) regions of central and peripheral necrosis that were typically absent in their control counterparts. In this experiment, MCA102 fibrosarcoma tumor sections were stained with H&E to determine blood vessel density (data not shown), which was quantified by manually counting 5 random high power fields (400×). FIG. 6D shows the corresponding mean (±SE; n=3) blood vessel densities normalized to GFP tumors from the H&E stained tumor sections (*, P<0.05).

Collectively, these findings establish FBLNs 3 and 5 as novel antagonists of tumor angiogenesis in vivo and, more importantly, indicate that these angiostatic activities may one be developed to prevent the growth and progression of human malignancies.

Summarizing the results of Examples 1-5, the present inventors have discovered that FBLN-3 is a novel angiogenesis antagonist that inhibits endothelial cell angiogenic sprouting by preventing their proliferation and invasion (FIG. 2C), and by reducing their activation of p38 MAPK stimulated by VEGF (FIG. 2E). Mechanistically, the present inventors establish FBLN-3, like FBLN-5, as a novel repressor of endothelial cell expression of MMP-2 and -3 and, conversely, as an inducer of TIMP-1, TIMP-3, and TSP-1 expression (FIG. 3). Moreover, like FBLN-5, FBLN-3 antagonized the angiogenic activities of bFGF both in vitro (FIG. 4) and in vivo (FIG. 5). Finally, the present inventors have provided the first demonstration that tumorigenesis is inhibited by FBLN-3, like FBLN-5, in part, through its ability to antagonize tumor angiogenesis (FIG. 6). These findings are especially important because FBLNs 3 and 5 inhibited angiogenesis and tumorigenesis in genetically normal mice, thereby excluding the possibility that altered angiogenesis arose as a consequence of defective elastogenesis (Nakamura et al., 2002; Yanagisawa et al., 2002) or other secondary effects associated with FBLN-deficiency. Indeed, the data provided herein indicates that the anti-angiogenic activities of FBLN-3, like FBLN-5, can be exploited as a cancer chemopreventive agent designed to combat tumor angiogenesis.

Another especially important finding of the present inventors' study concerned the nature of context-specific activities of FBLN-5 and, by extension, FBLN-3 (FIG. 6). For instance, one of the present inventors reported previously that FBLN-5 stimulates normal and malignant fibroblast proliferation, migration, and invasion (Schiemann et al.; U.S. patent application Publication No. 2004-0126788A1), while simultaneously inhibiting these activities in endothelial cells (Albig and Schiemann, 2004; U.S. patent application Publication No. 2004-0126788A1). In addition, the present inventor observed FBLN-5 to inhibit epithelial cell proliferation, as well as found tumorigenesis to inappropriately repress FBLN-5 expression in the vast majority of epithelial-derived human tumors (Schiemann et al., 2002; U.S. patent application Publication No. 2004-0126788A1). Tumors are essentially miniature organs comprised of malignant and normal cells, including fibroblasts, endothelial, and immune cells (Bissell and Radisky, 2001). Thus, given the context-specific activities of FBLN-5, it remained to be determined which cell type and biological activity is targeted predominantly by FBLN-5 (and FBLN-3) in developing tumor microenvironments. To this end, the present inventors show herein that despite their ability to enhance the apparent tumorigenicity of MCA102 fibrosarcoma cells in vitro (FIG. 6), FBLNs 3 and 5 both significantly decreased the mass of and blood vessel densities in developing MCA102 fibrosarcoma tumors in mice. As such, without being bound by theory, the present inventors propose that within the intricate context of the tumor microenviromnents, the ability of FBLNs 3 and 5 to promote angiostasis and to engender tumor suppressive microenvironments overrides their potential to enhance the growth and motility of cancer cells.

### Example 6

The following example shows that short FBLN-3 and long FBLN-3 differentially regulate endothelial cell proliferation, endothelial cell angiogenic sprouting, and endothelial cell invasion.

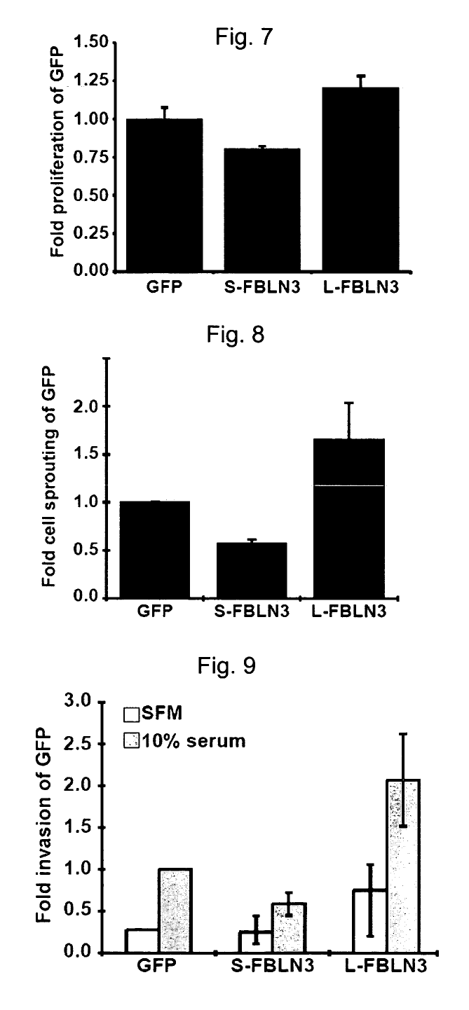

FIGS. 7-9 illustrate the results of an experiment in which the effects of short FBLN-3 and long FBLN-3 are compared in cell proliferation, cell sprouting, and tumor cell invasion experiments.

FIG. 7 shows that short and long Fibulin-3 molecules differentially regulate endothelial cell proliferation. The proliferation rates of control-, short form-, or long form-expressing Fibulin-3 MB114 cells were determined using a [³H]thymidine assay. The results show that Short Fibulin-3 inhibits endothelial cell DNA synthesis, while its long form counterpart stimulates endothelial cell proliferation.

FIG. 8 shows that short and long Fibulin-3 molecules differentially regulate endothelial cell angiogenic sprouting. Control-, short form-, or long form-expressing MB114 cells were allowed to tubulate on collagen gels for 5 days. Afterward, the quantity of invading angiogenic sprouts was determined by manual counting under a light microscope. The results demonstrate that short Fibulin-3 antagonizes endothelial cell angiogenic sprouting, while its long form counterpart induces endothelial cell sprouting.

FIG. 9 shows that short and long Fibulin-3 molecules differentially regulate endothelial cell invasion. Control-, short form-, and long form-Fibulin-3-expressing MB114 cells were allowed to invade synthetic Matrigel matrices in the absence or presence 10% serum as indicated. Afterward, the quantity of invading endothelial cells was determined by manual counting under a light microscope. The results show that short Fibulin-3 inhibits endothelial cell invasion, while its long form counterpart stimulates endothelial cell invasion.

### REFERENCES

Timpl et al. Nat Rev Mol Cell Biol 2003; 4:479-89.
Argraves et al. EMBO Rep 2003; 4:1127-31.
Balbona et al. J Biol Chem 1992; 267:20120-5.
Pan et al. J Cell Biol 1993; 123:1269-77.
Kowal et al. Circ Res 1999; 84:1166-76.
Nakamura et al. J Biol Chem 1999; 274:22476-83.
Schiemann et al. J Biol Chem 2002; 277:27367-77.
Nakamura et al. Nature 2002; 415:171-5.
Kapetanopoulos et al. Mol Genet Genomics 2002; 267:440-6.
Yanagisawa et al. Nature 2002; 415:168-71.
Zanetti et al. Mol Cell Biol 2004; 24:638-50.
Loeys et al. Hum Mol Genet 2002; 11:2113-8.
Markova et al. Am J Hum Genet 2003; 72:998-1004.
Kuang et al. Am J Physiol Lung Cell Mol Physiol 2003; 285:11147-152.
Jean et al. Am J Physiol Lung Cell Mol Physiol 2002; 282:L75-82.
Carmeliet P. Nat Med 2000; 6:389-95.
Carmeliet et al. Nature 2000; 407:249-57.
Kerbel et al. Nat Rev Cancer 2002; 2:727-39
Stupack et al. Oncogene 2003; 22:9022-9.
Albig et al. DNA Cell Biol 2004; (submitted).
Schiemann et al. Mol Biol Cell 2003; 14:3977-88.
Shannon et al. Biochim Biophys Acta 1987; 931:143-56.
Laiho et al. Cell 1990; 62:175-85.
Basolo et al. Int J Cancer 1994; 56:736-42.
Pepper M S. Cytokine Growth Factor Rev 1997; 8:21-43.
Pepper et al. Exp Cell Res 1993; 204:356-63.
Kanno et al. Oncogene 2000; 19:2138-46.
Gille et al. J Biol Chem 2001; 276:3222-30.
Rousseau et al. Oncogene 1997; 15:2169-77.
Hood et al. Nat Rev Cancer 2002; 2:91-100.
Schwartz et al. Nat Cell Biol 2002; 4:E65-8.
Ikegawa et al. Genomics 1996; 35:590-2.
Giltay et al. Matrix Biol 1999; 18:469-80.
Lecka-Czernik et al. Mol Cell Biol 1995; 15:120-8.
Hogan et al. Nat Rev Genet 2002; 3:513-23.
Yoshida et al. J Natl Cancer Inst 2000; 92:1717-30.
Blobe et al. N Engl J Med 2000; 342:1350-8.
Hanahan et al. Nat Med 1995; 1:27-31.
Stone et al. Nat Genet 1999; 22:199-202.
Marmorstein et al. Proc Natl Acad Sci USA 2002; 99:13067-72.
Matsumoto et al. Am J Ophthalmol 2001; 131:810-2.
Bell et al. J Cell Sci 2001; 114:2755-73.
Kahn et al. Am J Pathol 2000; 156:1887-900.
Kostka et al. Mol Cell Biol 2001; 21:7025-34.
Sasaki et al. EMBO J 1998; 17:4249-56.
Folkman et al. J Biol Chem 1992; 267:10931-4.
Terman et al. Cancer Metastasis Rev 1996; 15:159-63.
Inoki et al. FASEB J 2002; 16:219-21.
Kupprion et al. J Biol Chem 1998; 273:29635-40.
Lapis et al. Semin Cancer Biol 2002; 12:209-17.
Albig et al. DNA Cell Biol. June 2004; 23(6):367-79
U.S. patent application Publication No. 2004-0126788A1, published Jul. 1, 2004

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method for detecting tumorigenicity in a subject, comprising:
a) detecting a level of expression or activity of fibulin-3 (FBLN-3) in a test sample from a subject to be diagnosed;
b) comparing the level of expression or activity of FBLN-3 in the test sample to a baseline level of FBLN-3 expression or activity established from a control sample; and
c) determining whether there is a difference between the levels of FBLN-3 expression;
wherein detection of a statistically significant difference in the level of FBLN-3 expression or activity in the test sample, as compared to the baseline level of FBLN-3 expression or biological activity, indicates a difference in tumorigenicity in the test sample as compared to in the control sample.

2. The method of claim 1, wherein the FBLN-3 is short FBLN-3.

3. The method of claim 2, wherein the FBLN-3 consists of amino acids 107-493 of SEQ ID NO:2.

4. The method of claim 1, wherein the FBLN-3 is long FBLN-3.

5. The method of claim 4, wherein the FBLN-3 comprises SEQ ID NO:2.

6. The method of claim 1, wherein the step of detecting comprises detecting FBLN-3 mRNA transcription by cells in the test sample.

7. The method of claim 6, wherein the step of detecting is by a method selected from the group consisting of polymerase chain reaction (PCR), reverse transcriptase-PCR (RT-PCR), in situ hybridization, Northern blot, sequence analysis, gene microarray analysis, and detection of a reporter gene.

8. The method of claim 1, wherein the step of detecting comprises detecting FBLN-3 protein in the test sample.

9. The method of claim 8, wherein the step of detecting is by a method selected from the group consisting of immunoblot, enzyme-linked immunosorbant assay (ELISA), radioimmunoassay (RIA), immunoprecipitation, immunohistochemistry and immunofluorescence.

10. The method of claim 1, wherein the step of detecting comprises detecting FBLN-3 biological activity in the test sample.

11. The method of claim 10, wherein the step of detecting comprises measuring proliferation of cells expressing FBLN-3, detecting DNA synthesis in cells expressing FBLN-3, detecting MAP kinase activity in cells expressing FBLN-3, detecting MAP kinase activity in the presence of the test sample, and measuring migration and invasion ability of fibroblasts expressing FBLN-3; detecting the ability of FBLN-3 to regulate vascular endothelial growth factor (VEGF) signaling; detecting the ability of FBLN-3 to regulate matrix metalloproteinase (MMP) expression and activity; and detecting the ability of FBLN-3 to regulate tissue inhibitor of metalloproteinase (TIMP) expression.

12. The method of claim 1, wherein the test sample is from a source selected from the group consisting of: breast, kidney, ovary, colon, and uterus.

13. The method of claim 1, wherein the test sample is a fibroblast cell sample.

14. The method of claim 1, wherein detection of a statistically significant difference in the level of FBLN-3 expression or activity in the test sample as compared to the baseline level, with a confidence of p<0.05, indicates that the cells in the test sample have a difference in tumorigenicity as compared to the control sample.

15. The method of claim 1, wherein a detection of an at least about 10% difference in the level of FBLN-3 expression or activity in the test sample as compared to the baseline level, with a confidence of p<0.05, indicates that the cells in the test sample have a difference in tumorigenicity as compared to the control sample.

16. The method of claim 1, wherein a detection of an at least about 1.5 fold difference in the level of FBLN-3 expression or activity in the test sample as compared to the baseline level, with a confidence of p<0.05, indicates that the cells in the test sample have a difference in tumorigenicity as compared to the control sample.

17. The method of claim 1, wherein the test sample is from a patient being diagnosed for cancer and wherein the baseline level is established from a negative control sample that is non-tumorigenic, and wherein detection of a statistically significant difference in the level of FBLN-3 expression or activity in the test sample, as compared to the baseline level of FBLN-3 expression or biological activity in the negative control sample, indicates the presence of tumor cells in the test sample.

18. The method of claim 17, wherein, when the FBLN-3 expression or biological activity detected in step (a) is statistically significantly different from the baseline level of FBLN-3 expression or activity, the method further comprises:
d) comparing the FBLN-3 expression or activity of the test sample as detected in step (a) to levels of FBLN-3 expression or activity from a panel of tumor-positive control samples, wherein each of the tumor-positive control samples is correlated with a different stage of tumor development; and,
e) identifying a level of FBLN-3 expression or activity from one of the tumor-positive control samples which is statistically significantly most similar to the level of FBLN-3 expression or biological activity detected in step (a), to diagnose a stage of tumor development in the patient.

19. The method of claim 17, wherein FBLN-3 is short FBLN-3, wherein the test sample is not a fibroblast cell sample, and wherein a decrease in the level of short FBLN-3 expression or activity of the test sample as compared to the baseline level of expression or activity indicates the presence of tumor cells in the test sample.

20. The method of claim 1, wherein the test sample is from a patient who is known to have cancer, and wherein the baseline level comprises a first level of FBLN-3 expression or activity from a previous tumor cell sample from the patient and a second level of FBLN-3 expression or activity established from a negative control cell sample that is non-tumorigenic;
wherein a statistically significant change in the level of FBLN-3 expression or activity in the test sample toward the baseline level established from the negative control cell sample, as compared to the baseline level of expression or activity from the previous tumor cell sample, indicates a reduction in tumor cells in the test sample as compared to the previous tumor cell sample.

21. The method of claim 20, wherein the method further comprises a step (d) of modifying cancer treatment for the patient if no statistically significant change in the level of FBLN-3 expression or activity in the test sample toward the baseline level established from the negative control cell sample is detected.

22. The method of claim 1, wherein the baseline level is established by a method selected from the group consisting of:
i) establishing a baseline level of FBLN-3 expression or activity in an autologous control sample from the patient, wherein the autologous sample is from a same cell type, tissue type or bodily fluid type as the test sample of step (a);
ii) establishing a baseline level of FBLN-3 expression or activity from at least one previous detection of FBLN-3 expression or activity in a previous test sample from the patient, wherein the previous test sample was of a same cell type, tissue type or bodily fluid type as the test sample of step (a); and,
iii) establishing a baseline level of FBLN-3 expression or activity from an average of control samples of a same cell type, tissue type or bodily fluid type as the test sample of step (a), the control samples having been obtained from a population of matched individuals.

23. A test kit for assessing the tumorigenicity of cells in a patient, comprising:
a) a means for detecting FBLN-3 expression or activity in a test sample; and
b) a means for detecting a control marker characteristic of a cell or tissue type that is in the test sample or that is secreted into the test sample by the cell or tissue.

24. The test kit of claim 23, wherein the means of (a) is selected from the group consisting of: a hybridization probe of at least about 8 nucleotides that hybridizes under stringent hybridization conditions to a nucleic acid molecule encoding FBLN-3 or a fragment thereof; an oligonucleotide primer for amplification of mRNA encoding FBLN-3 or a fragment thereof; and an antibody that selectively binds to FBLN-3.

25. The test kit of claim 23, wherein the means of (b) is selected from the group consisting of: a hybridization probe of at least about 8 nucleotides that hybridizes under stringent hybridization conditions to a nucleic acid molecule encoding the control marker or a fragment thereof; an oligonucleotide primer for amplification of mRNA encoding the control marker or a fragment thereof; and an antibody that selectively binds to the control marker.

26. The test kit of claim 23, wherein the means of (a) and (b) are suitable for use in a method of detection selected from the group consisting of immunohistochemistry and immunofluorescence.

27. A method to identify a compound useful for inhibition of tumor growth or malignancy, comprising:
a) detecting an initial level of FBLN-3 expression or activity in a tumor cell or soluble product derived therefrom;
b) contacting the tumor cell or soluble product with a test compound;
c) detecting a level of FBLN-3 expression or activity in the tumor cell or soluble product derived therefrom after contact of the tumor cell with the compound; and,
d) selecting a compound that changes the level of FBLN-3 expression or activity in the tumor cell or soluble product therefrom, as compared to the initial level of FBLN-3 expression or activity, toward a baseline level of FBLN-3 expression or activity established from a non-tumor cell, wherein the selected compound is predicted to be useful for inhibition of tumor growth or malignancy.

28. A method to identify a compound that regulates angiogenesis, comprising:
a) detecting an initial level of FBLN-3 expression or activity in a cell or soluble product derived therefrom;
b) contacting the cell or soluble product with a test compound;
c) detecting a level of FBLN-3 expression or activity in the cell or soluble product derived therefrom after contact of the cell with the compound; and,
d) selecting a compound that changes the level of FBLN-3 expression or activity in the cell or soluble product therefrom, as compared to in the absence of the compound or as compared to the initial level of FBLN-3 expression or activity, as a compound that regulates angiogenesis.

29. The method of claim 28, wherein the FBLN-3 is long FBLN-3, and wherein step (d) comprises selecting an agent that increases the expression or activity of long FBLN-3 as an agent that promotes angiogenesis or selecting an agent that decreases the expression or activity of long FBLN-3 as an agent that inhibits angiogenesis.

30. The method of claim 28, wherein the FBLN-3 is short FBLN-3, and wherein step (d) comprises selecting an agent that increases the expression or activity of short FBLN-3 as an agent that inhibits angiogenesis or selecting an agent that decreases the expression or activity of short FBLN-3 as an agent that promotes angiogenesis.

31. A method to regulate angiogenesis in a tissue of a subject, comprising regulating the expression or biological activity of FBLN-3 in the cells of the tissue.

32. The method of claim 31, wherein the method inhibits angiogenesis in the tissue of the subject, and wherein the method comprises increasing the expression or biological activity of short FBLN-3 in the cells of the tissue, or decreasing the expression or biological activity of long FBLN-3 in the cells of the tissue.

33. The method of claim 32, wherein the step of increasing the expression or activity of short FBLN-3 comprises administering short FBLN-3 or a biologically active homologue or analog thereof to the patient.

34. The method of claim 33, comprising expressing a recombinant nucleic acid molecule encoding short FBLN-3 or a homologue thereof in the tissue of the patient.

35. The method of claim 31, wherein the method promotes angiogenesis in a tissue of a patient, and wherein the method comprises decreasing the expression or biological activity of short FBLN-3 in the cells of the tissue, or increasing the expression or biological activity of long FBLN-3 in the cells of the tissue.

36. The method of claim 35, wherein the step of increasing the expression or activity of long FBLN-3 comprises administering long FBLN-3 or a biologically active homologue or analog thereof to the patient.

37. The method of claim 36, comprising expressing a recombinant nucleic acid molecule encoding long FBLN-3 or a homologue thereof in the tissue of the patient.

38. A method to reduce tumorigenicity in a patient, comprising regulating the expression or biological activity of FBLN-3 in tumor cells of the patient.

39. The method of claim 38, wherein the tumor cells are from a tissue selected from the group consisting of: breast, ovary, kidney, colon, and uterus.

40. The method of claim 38, comprising administering short FBLN-3 or a biologically active homologue or analog thereof to the patient.

41. The method of claim 38, comprising inhibiting the expression or biological activity of long FBLN-3 in the tumor cells.

42. The method of claim 38, comprising expressing a recombinant nucleic acid molecule encoding short FBLN-3 or a homologue thereof in the tissue of the patient.

43. A method to reduce tumorigenicity of a fibrosarcoma in a patient, comprising regulating the expression or biological activity of FBLN-3 in fibrosarcoma cells of the patient.

44. The method of claim 43, comprising decreasing the expression or biological activity of short FBLN-3 in fibrosarcoma cells of the patient.
